(19) United States
(12) Patent Application Publication
(10) Pub. No.: US 2017/0226571 A1
(43) Pub. Date: Aug. 10, 2017

(54) System and Method for Highly-Multiplexed, Label-Free Detection of Analytes Using Optical Tags

(71) Applicant: TruTag Technologies, Inc., Kapolei, HI (US)

(72) Inventors: Hod Finkelstein, Berkeley, CA (US); Tim Learmonth, Berkeley, CA (US)

(21) Appl. No.: 15/415,685

(22) Filed: Jan. 25, 2017

Related U.S. Application Data
(60) Provisional application No. 62/365,594, filed on Jul. 22, 2016.
(60) Provisional application No. 62/311,649, filed on Mar. 22, 2016.
(60) Provisional application No. 62/286,849, filed on Jan. 25, 2016.

(51) Int. Cl. C12Q 1/68; G01N 33/543; G01N 21/64

(52) U.S. Cl. CPC C12Q 1/6837; G01N 21/6428; G01N 33/54306; C12Q 1/6825; G01N 33/54366; G01N 2021/6441

(57) ABSTRACT

Provided herein are compositions, systems, and methods for performing a biological or chemical analysis of a sample using encoded functionalized optical tags. These optical tags can generate a unique spectral signature correlated with the identity of a probe bound to the optical tag, and a state of the interaction of the probe with an analyte. Also provided herein are methods of generating encoded functionalized optical tags.

### CROSS-REFERENCE TO RELATED APPLICATIONS

This application claims the benefit of U.S. Provisional Patent Application No. 62/286,849, filed Jan. 25, 2016, U.S. Provisional Patent Application No. 62/311,649, filed Mar. 22, 2016, and U.S. Provisional Patent Application No. 62/365,594, filed Jul. 22, 2016, the disclosures of which are incorporated herein by reference.

### FIELD OF THE INVENTION

The present invention is directed to compositions and methods for detection of analytes in multiplexed reactions using optical tags to facilitate label-free detection.

### BACKGROUND OF THE INVENTION

In order to simultaneously detect multiple analytes, labels have traditionally been used to determine the identity and presence of an analyte as it reacted with a probe. However, the use of labels, such as fluorophores, in a detection process can affect the properties of an analyte, leading to undesirable and unanticipated interactions that can compromise data and lead to false conclusions. In addition fluorophores are also costly, their attachment to an analyte is time-consuming, and they require sophisticated and expensive instruments for detection.

Although label-free detection has been achieved, it requires expensive components, is time consuming, and has not been demonstrated for multiplexed detection for large numbers of analytes. Some label free methods of detection use a shift in resonance energy of plasmonic waves to detect an incorporation on a substrate. Such methods require a coherent light source, very fine optical alignment in order to induce the plasmonic wave, and expensive substrates, typically gold. Furthermore, in order to multiplex targets, multiple substrates need to be created, each with its own optically-aligned light source. Another label-free method uses a shift in the resonant wavelength of a ring resonator upon successful incorporation of a target onto the surface of the ring resonator. This method requires expensive optics with a coherent source in order to couple into an array of waveguides (or a very lossy incoherent source), expensive lithography to create the resonators, and very fine spectroscopes to measure fine shifts in wavelength. Moreover, the available surface area of the ring resonator limits the sensitivity of such devices. These label-free detection techniques are limited with respect to the number of targets that can be simultaneously detected on an array of resonators.

Therefore, there is an unmet need in the art for a highly multiplexed, label-free method to identify a set of target analytes in a sample quickly, robustly and inexpensively.

### SUMMARY OF THE INVENTION

The instant invention is based, at least in part, on the discovery of a new label-free method of detecting reaction of target molecules with functionalized optical tags, by observing a shift in a spectral signature comprising, e.g., a rugate or Bragg peak, generated upon illumination of the optical tag. As used herein, a “shift” in a spectral signature refers to any observed change in the spectral signature.

Provided herein, according to some embodiments, is a method of detecting an analyte suspected of being present in a sample, comprising: providing a substrate comprising an optical tag immobilized on the surface of the substrate, wherein said optical tag is bound to a probe, wherein said optical tag comprises a plurality of pores, and wherein each of said plurality of pores comprises a modulated pore structure; contacting said substrate with a sample suspected of comprising an analyte, wherein the probe is capable of binding specifically to the analyte; exposing said optical tag to electromagnetic radiation to generate a sample spectral signature comprising at least one spectral feature that is a function of the modulated pore structure of the optical tag across a range of wavelengths; detecting said sample spectral signature; and comparing said sample spectral signature with a reference spectral signature to detect said analyte in said sample.

In some embodiments, the method of detecting an analyte suspected of being present in a sample further comprises exposing said immobilized optical tag to electromagnetic radiation prior to contacting said substrate with said sample to generate a reference spectral signature comprising at least one spectral feature that is a function of the modulated pore structure of the optical tag across a range of wavelengths; detecting said reference spectral signature; and storing said reference spectral signature in a memory.

In some embodiments, the modulated pore structure across the plurality of pores in the optical tag is substantially similar. In some embodiments, the reference spectral signature is determined from a functionalized or non-functionalized optical tag before contact with said sample. In some embodiments, the reference spectral signature is directly measured or statistically determined. In some embodiments, the statistically determined reference spectral signature is a function of a measured spectral signature from one or more optical tags with the same modulated pore structure, or is a function of the programmed properties of the modulated pore structure.

In some embodiments, the at least one spectral feature of said reference or sample spectral signature is a rugate peak. In some embodiments, the at least one spectral feature of said reference or sample spectral signature is a Bragg peak. In some embodiments, the reference or sample spectral signature further comprises a Fabry-Perot spectral response. In some embodiments, the spectral feature is a peak or a trough.

In some embodiments, the sample or reference spectral signature is linked to said optical tag by a spectral feature selected from the group consisting of: a unique peak number, a unique peak placement, a unique peak phase, a unique peak amplitude, a unique trough number, a unique trough placement, a unique trough phase, and a unique trough amplitude.

In some embodiments, comparing the sample spectral signature with the reference spectral signature comprises identifying the presence or absence of a spectral signature shift between the sample spectral signature and the reference spectral signature. In some embodiments, the detection of the spectral signature shift indicates the presence of the analyte in said sample. In some embodiments, the spectral signature shift comprises a shift in a peak placement, peak phase, peak number, or peak amplitude. In some embodiments, the spectral signature shift comprises a shift in a trough placement, trough phase, trough number, or trough amplitude.

In some embodiments, the optical tag comprises silica or silicon. In some embodiments, the optical tag is partially oxidized or fully oxidized. In some embodiments, the optical tag comprises a non-silica dielectric. In some embodiments, the optical tag comprises a stack of dielectric layers.

In some embodiments, the optical tag has a porosity of from 60 to 95%. In some embodiments, the plurality of pores is sufficiently large to facilitate entry of said target analyte into said pores while excluding larger non-target molecules.

In some embodiments, the optical tag comprises a silica linker. In some embodiments, the linker is an organofunctional alkoxysilane molecule. In some embodiments, the probe is bound to the surface of the optical tag. In some embodiments, the probe is bound within one of said plurality of pores of the optical tag.

In some embodiments, the probe comprises an oligonucleotide or a polypeptide. In some embodiments, the probe comprises a receptor, an aptamer, an antibody or an antibody fragment. In some embodiments, the probe comprises a layer of amino acids across the surface of the optical tag.

In some embodiments, the sample is liquid, air, or vapor, or part of a liquid assay or Non-Invasive Prenatal Test (NIPT) test.

In some embodiments, the substrate comprises glass, paper, plastic, a polymer, or any combination thereof. In some embodiments, the substrate comprises a plurality of unique optical tags immobilized on the surface of the substrate.

In some embodiments, each of said unique optical tags is configured to generate a unique spectral signature comprising at least one peak or at least one trough that is a function of the modulated pore structure of the optical tag. In some embodiments, the modulated pore structure is unique for each of said unique optical tags. In some embodiments, the identity of said probe is correlated with said unique optical tag.

In some embodiments, the detection of said reference or sample spectral signature comprises detecting an intensity at 20 or more, 30 or more, 40 or more, 50 or more, 60 or more, 70 or more, 80 or more, 90 or more, or 100 or more different narrow wavelength bands from a single one of said optical tags on the substrate. In some embodiments, the detection of said reference or sample spectral signature is performed using a device comprising an interferometer.

In some embodiments, the interferometer is a tunable Fabry-Perot interferometer or a Michelson type interferometer. In some embodiments, the detection of said reference or sample spectral signature comprises obtaining a wide spectral range from a field of view of about 100 mm². In some embodiments, the detection of said reference or sample spectral signature comprises placing said substrate in a portable device capable of obtaining a wide spectral range of said spectral signature. In some embodiments, the portable device displays an identity of the analyte upon detection.

In some embodiments, the analyte does not comprise a detection label. In some embodiments, the probe does not comprise a detection label. In some embodiments, the method is label-free.

In some embodiments, the optical tag has a diameter, length, width, depth or height that is less than or equal to a millimeter. In some embodiments, the optical tag has a diameter, length, width, depth or height that is less than or equal to 0.1 mm, 0.2 mm, 0.3 mm, 0.4 mm, 0.5 mm, 0.6 mm, 0.7 mm, 0.8 mm, 0.9 mm, or 1.0 mm. In some embodiments, the optical tag has a diameter, length, width, depth or height that is less than or equal to 0.01 mm, 0.02 mm, 0.03 mm, 0.04 mm, 0.05 mm, 0.06 mm, 0.07 mm, 0.08 mm, 0.09 mm, or 0.1 mm. In some embodiments, the optical tag has a diameter, length, width, depth or height that is less than or equal to 0.1 μm, 0.2 μm, 0.3 μm, 0.4 μm, 0.5 μm, 0.6 μm, 0.7 μm, 0.8 μm, 0.9 μm, or 1.0 μm.

In some embodiments, detecting said analyte in said sample comprises determining the presence or absence of said analyte in said sample. In some embodiments, detecting said analyte in said sample comprises determining a property of said analyte in said sample. In some embodiments, the property is a concentration of said analyte, a binding affinity of said analyte to said probe, or a specific activity of said analyte. In some embodiments, the substrate comprises a plurality of said optical tags, and wherein said concentration is determined from a proportion of said plurality of optical tags generating a shifted spectral signature, or is determined from a change in the average spectral signature for the plurality of optical tags.

In some embodiments, the method of detecting an analyte suspected of being present in a sample further comprises performing a nucleic acid amplification reaction of said analyte before contacting said substrate with said sample, wherein said analyte is a polynucleotide. In some embodiments, the nucleic acid amplification reaction is a polymerase chain reaction or an isothermal amplification reaction. In some embodiments, the isothermal amplification reaction is a recombinase polymerase amplification reaction.

In some embodiments, the method of detecting an analyte suspected of being present in a sample further comprises purifying said analyte from said sample using magnetic beads. In some embodiments, the method of detecting an analyte suspected of being present in a sample further comprises exposing said sample to an electric field during contact with said substrate. In some embodiments, the method of detecting an analyte suspected of being present in a sample further comprises washing said substrate to remove non-specifically bound molecules. In some embodiments, the substrate is washed with buffer or air.

Also provided herein is a method of detecting one or more analytes suspected of being present in a sample, comprising: providing a plurality of optical tags each bound to at least one probe, wherein said plurality of optical tags each comprise a plurality of pores comprising a modulated pore structure; contacting the plurality of optical tags with a sample suspected of comprising one or more analytes, wherein each probe is capable of binding specifically to one or more analytes or families of analytes; immobilizing the plurality of optical tags on a surface of a substrate; exposing each of the plurality of optical tags to electromagnetic radiation to generate a sample spectral signature for each of the plurality of optical tags, wherein the sample spectral signature comprises at least one spectral feature that is a function of the modulated pore structure of the optical tag across a range of wavelengths; detecting said sample spectral signature for each of the plurality of optical tags; and comparing said sample spectral signature with a reference spectral signature for each of said plurality of optical tags to detect said analyte in said sample.

In some embodiments, contacting the plurality of the optical tags with the sample is performed prior to immobilizing the plurality of optical tags on the surface of the substrate. In some embodiments, immobilizing the plurality of optical tags on the surface of the substrate is performed prior to contacting the plurality of optical tags with the sample.

In some embodiments, the modulated pore structure across the plurality of pores in each unique optical tag from said plurality of optical tags is substantially similar. In some embodiments, the reference spectral signature is determined from at least one functionalized or non-functionalized optical tag from said plurality of optical tags before contact with said sample.

In some embodiments, the reference spectral signature is directly measured or statistically determined. In some embodiments, the statistically determined reference spectral signature is a function of a measured spectral signature from one or more optical tags with the same modulated pore structure as an optical tag from said plurality of optical tags, or is a function of the programmed properties of the modulated pore structure of said optical tag from said plurality of optical tags.

In some embodiments, the at least one spectral feature of said reference or sample spectral signature is a rugate peak. In some embodiments, the at least one spectral feature of said reference or sample spectral signature is a Bragg peak. In some embodiments, the reference or sample spectral signature further comprises a Fabry-Perot spectral response. In some embodiments, the spectral feature is a peak or a trough.

In some embodiments, the sample or reference spectral signature is linked to a unique optical tag from said plurality of optical tags by a spectral signature feature selected from the group consisting of: a unique peak number, a unique peak placement, a unique peak phase, a unique peak amplitude, a unique trough number, a unique trough placement, a unique trough phase, and a unique trough amplitude.

In some embodiments, comparing the sample spectral signature with the reference spectral signature comprises identifying the presence or absence of a spectral signature shift between the sample spectral signature and the reference spectral signature. In some embodiments, detection of the spectral signature shift indicates the presence of the analyte in said sample. In some embodiments, the spectral signature shift comprises a shift in a peak placement, peak phase, peak number, or peak amplitude. In some embodiments, the spectral signature shift comprises a shift in a trough placement, trough phase, trough number, or trough amplitude.

In some embodiments, the plurality of optical tags comprise silica or silicon. In some embodiments, the plurality of optical tags are partially oxidized or fully oxidized. In some embodiments, the plurality of optical tags comprises a non-silica dielectric. In some embodiments, at least one of the plurality of optical tags comprises a stack of dielectric layers.

In some embodiments, the plurality of optical tags have a porosity of from 60 to 95%. In some embodiments, the plurality of pores is sufficiently large to facilitate entry of said target analyte into said pores while excluding larger non-target molecules.

In some embodiments, the plurality of optical tags comprise a silica linker. In some embodiments, the linker is an organofunctional alkoxysilane molecule.

In some embodiments, the at least one probe comprises an oligonucleotide or a polypeptide. In some embodiments, the at least one probe comprises a receptor, an aptamer, an antibody or an antibody fragment. In some embodiments, the at least one probe comprises a layer of amino acids across the surface of the optical tag.

In some embodiments, the sample is liquid, air, or vapor. In some embodiments, contacting the plurality of optical tags with the sample comprises mixing the plurality of optical tags with the sample in a solution or in a gaseous environment. In some embodiments, the method of detecting one or more analytes suspected of being present in a sample further comprises removing said plurality of optical tags from said solution via centrifugation, filtration, or electrophoresis.

In some embodiments, the substrate comprises glass, paper, plastic, a polymer, or any combination thereof. In some embodiments, the substrate comprises a filter comprising pores that are smaller than the size of most of the plurality of optical tags.

In some embodiments, the plurality of optical tags comprises a plurality of unique optical tags. In some embodiments, each of said unique optical tags is configured to generate a unique spectral signature comprising at least one peak or trough that is a function of the modulated pore structure of the optical tag. In some embodiments, the modulated pore structure is unique for each of said unique optical tags. In some embodiments, the identity of said at least one probe is correlated with each of said unique optical tags.

In some embodiments, the detection of said reference or sample spectral signature comprises detecting an intensity at 20 or more, 30 or more, 40 or more, 50 or more, 60 or more, 70 or more, 80 or more, 90 or more, or 100 or more different narrow wavelength bands from a single one of said optical tags on the substrate.

In some embodiments, detection of said reference or sample spectral signature is performed using a device comprising a tunable Fabry-Perot interferometer. In some embodiments, detection of said reference or sample spectral signature comprises obtaining a wide spectral range from a field of view of about 100 mm². In some embodiments, detection of said reference or sample spectral signature comprises placing said substrate in a portable device capable of obtaining a wide spectral range of said spectral signature. In some embodiments, the portable device displays an identity of the analyte upon detection.

In some embodiments, the analyte does not comprise a detection label. In some embodiments, the probe does not comprise a detection label. In some embodiments, the method is label-free

In some embodiments, each of said plurality of optical tags has a diameter, length, width, depth or height that is less than or equal to a millimeter (mm). In some embodiments, each of said plurality of optical tags has a diameter, length, width, depth or height that is less than or equal to 0.1 mm, 0.2 mm, 0.3 mm, 0.4 mm, 0.5 mm, 0.6 mm, 0.7 mm, 0.8 mm, 0.9 mm, or 1.0 mm. In some embodiments, each of said plurality of optical tags has a diameter, length, width, depth or height that is less than or equal to 0.01 mm, 0.02 mm, 0.03 mm, 0.04 mm, 0.05 mm, 0.06 mm, 0.07 mm, 0.08 mm, 0.09 mm, or 0.1 mm. In some embodiments, each of said plurality of optical tags has a diameter, length, width, depth or height that is less than or equal to 0.1 μm, 0.2 μm, 0.3 μm, 0.4 μm, 0.5 μm, 0.6 μm, 0.7 μm, 0.8 μm, 0.9 μm, or 1.0 μm.

In some embodiments, detecting said one or more analytes in said sample comprises determining the presence or absence of said one or more analytes in said sample. In some embodiments, detecting said one or more analytes in said sample comprises determining a property of said one or more analytes in said sample. In some embodiments, the property is a concentration of said one or more analytes analyte, a binding affinity of said one or more analytes to one or more of said probes, or a specific activity of said one or more analytes. In some embodiments, the concentration is determined from a proportion of said plurality of optical tags generating a shifted sample spectral signature, or is determined from a change in the average spectral signature for the plurality of optical tags.

In some embodiments, the method of detecting one or more analytes suspected of being present in a sample further comprises performing a nucleic acid amplification reaction of said analyte before contacting said substrate with said sample, wherein said analyte is a polynucleotide. In some embodiments, the nucleic acid amplification reaction is a polymerase chain reaction or an isothermal amplification reaction. In some embodiments, the isothermal amplification reaction is a recombinase polymerase amplification reaction.

In some embodiments, the method of detecting one or more analytes suspected of being present in a sample further comprises purifying said analyte from said sample using magnetic beads. In some embodiments, the method of detecting one or more analytes suspected of being present in a sample further comprises exposing said sample to an electric field during contact with said substrate. In some embodiments, the method of detecting one or more analytes suspected of being present in a sample further comprises washing said substrate to remove non-specifically bound molecules. In some embodiments, the substrate is washed with buffer or air.

Also provided herein, according to some embodiments, is a composition comprising a substrate comprising a plurality of unique optical tags immobilized on the surface of the substrate, wherein each unique optical tag comprises a plurality of pores comprising a unique modulated pore structure, and wherein each unique optical tag is bound to a probe associated with said unique modulated pore structure.

In some embodiments, the probe is capable of binding specifically to an analyte. In some embodiments, the plurality of pores are sufficiently large to facilitate entry of said analyte into said pores while excluding larger non-target molecules.

In some embodiments, the modulated pore structure throughout each of said unique optical tags is substantially similar. In some embodiments, each of said unique optical tags is configured to generate a spectral signature that is detectably different from other unique optical tags when exposed to electromagnetic radiation. In some embodiments, the spectral signature comprises at least one rugate or Bragg peak.

In some embodiments, the plurality of unique optical tags comprise silica or silicon. In some embodiments, the plurality of unique optical tags is partially or fully oxidized. In some embodiments, the plurality of unique optical tags comprise a non-silica dielectric. In some embodiments, the plurality of unique optical tags have a porosity of from 60 to 95%.

Also provided herein, according to some embodiments, is a method of a making a composition comprising a plurality of functionalized optical tags immobilized on a substrate, comprising: preparing a plurality of unique optical tags comprising a plurality of pores comprising a unique modulated pore structure; binding at least one probe to each of said plurality of unique optical tags, wherein said probe is correlated with said unique modulated pore structure; mixing said plurality of unique optical tags; and immobilizing said plurality of unique optical tags on the surface of a substrate.

In some embodiments, also provided herein is a diagnostic system for detecting the presence or absence of an analyte in a sample, comprising: an optical tag bound to a probe, wherein said optical tag comprises a modulated pore structure, wherein said optical tag has been contacted with a sample suspected of containing an analyte; and a reader device comprising one or more broadband sources configured to illuminate said optical tag; a detector configured to detect reflected or transmitted light comprising a spectral response from said optical tag, wherein said spectral response comprises at least one spectral feature that is a function of the modulated pore structure of the optical tag across a range of wavelengths; and a display configured to display results of the detection performed by the detector, the results indicating the presence or absence of the analyte in the sample.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1 is a schematic diagram depicting a procedure for functionalization of optical tags of the disclosure.

FIG. 2 is a schematic diagram depicting a procedure for a sample binding to a composition of the disclosure.

FIG. 3 is a schematic diagram depicting a procedure for sample analysis flow with a factory optical tag spectral reference.

FIG. 4 is a schematic diagram depicting a procedure for Measurement flow without a factory optical tag spectral reference.

FIG. 5 is a graph illustrating an optical shift due to binding of a target with an optical tag.

FIG. 6 is a schematic diagram depicting exemplary signals generated before, during and after a binding event of a target molecule to a reactive moiety attached via a coating material to a porous silica substrate.

FIG. 7A is a diagram of an example diagnostic system including a substrate with optical tags and a reader for reading the substrate.

FIG. 7B is a network diagram of an example system environment including the diagnostic system in communication with one or more client devices and one or more servers via a network.

FIG. 8 is a graph showing the shift in feature wavelength between non-complementary target exposure and complementary target exposure for all features in a spectral waveform (left), and an example of a spectral waveform before functionalization and after complementary exposure, showing the shift in wavelength (right).

### DETAILED DESCRIPTION

The details of various embodiments of the invention are set forth in the description below. Other features, objects, and advantages of the invention will be apparent from the description and the drawings, and from the claims.

Introduction

The compositions, systems and methods disclosed herein are designed to provide a label-free method of high-throughput and multiplexed detection of analytes in a sample. By using the compositions, systems, and methods provided herein, one can detect the presence, concentration, and physical aspects of one or a large number of target molecules interacting with one or more optical tags efficiently and without requiring the use of any external labels, such as fluorophores, to identify the interaction signal. Instead, we provide herein a system that can detect the presence and other properties associated with a target molecule by observing and analyzing one or more optical signatures each associated with one or more target molecules.

In some embodiments, disclosed herein are compositions and methods for producing and reading a plurality of porous optical tags that produce a distinct spectral signature when contacted with electromagnetic radiation. Porous optical tags of the disclosure can comprise silica, a non-silica, a pure or partially-pure dielectric, or a partially-oxidized silicon. In preferred embodiments, the porous optical tags further comprise a reactive moiety capable of reacting with (e.g., binding) at least one target entity (e.g., a target biomolecule).

Specifically, in some embodiments, disclosed herein is a composition comprising a substrate and a plurality of optical tags, wherein the optical tags comprise at least one surface and a plurality of pores with a modulated pore structure capable of producing a spectral signature, e.g., comprising at least one rugate or Bragg spectral peak or trough, or both, when exposed to a light source. These optical tags are functionalized and bound to a reactive moiety capable of reacting with at least one target molecule. Each unique optical tag (i.e., each tag with a unique modulated pore structure or modulated pore structure) gives rise to a unique optical signature upon exposure to electromagnetic radiation. This unique optical signature is correlated with at least one probe comprising the reactive moiety bound to the pore. Thus, the substrate can be used for multiplexed detection wherein at least two unique optical tags in the plurality comprise a different reactive moiety each capable of reacting with a respective unique target molecule. The unique optical signature generated by each unique optical tag when exposed to a source of illumination can then be used to determine the identity of the reactive moiety and or target molecule that reacts with the reactive moiety.

Also provided herein is a method of producing and reading a plurality of encoded optical tags used in an assay, e.g., for biological or chemical analysis. The method can include providing a plurality of encoded porous optical tags comprising a modulated pore structure and functionalized to comprise probes capable of reacting with a target bound to the optical tag. The probes facilitate the detection of the presence or absence of a respective target analyte on or within the vicinity of the optical tag.

In some embodiments, provided herein is a method for applying and immobilizing a collection of such optical tags on a substrate. In some embodiments, provided herein is a method for introducing a sample suspected of comprising one or more analytes onto the substrate comprising the immobilized optical tags. In some embodiments, provided herein is a system for efficiently reading spectral signatures from said substrate and identifying the presence or absence of bound analytes. In some embodiments, a method of detection using the functionalized optical tags can include an algorithm to detect the relative abundance of the analytes in the sample. Also provided herein, according to some embodiments, is a system that facilitates the transmission and sharing of the analysis results. Compositions and methods of the disclosure may be used in an assay for biological or chemical analysis.

In some embodiments, the substrate is a test strip or other device onto which a sample can be placed, such as a sample from a user or individual (e.g., body fluid, such as saliva), The test strip can be read by a reader or display device, such as a diagnostic device that has hyperspectral imaging capabilities. The reader or display device can identify optical tags whose spectrum has shifted due to a binding event that has occurred on the strip. The device can read the optical spectral code of the optical tags and detect the analyte (e.g., a pathogen) in the sample. In some embodiments, the device displays the result of the test, though in other cases the device communicates via a network to a user's computer or mobile device to display the result of the test. As one example, the result may indicate that a particular flu pathogen has been detected in the user's sample. In some embodiments, the reader or display device communicates via a network to other devices, such as devices of the user's physician or other medical personnel, the user's family or friends, a school attended by a user, or individuals or institutions to whom the user might wish to provide the results.

Functionalized Optical Tags

Provided herein are functionalized optical tags that comprise a probe bound to the surface of the optical tag, wherein the probe is capable of binding to a target of interest. The optical tag acts as an identifier of the probes bound to its surface by having a series of structures that generate a unique, readable spectral response that is observed by analyzing reflected or transmitted light either from, or through, these optical tags, respectively called “reflectance spectra” and “transmission spectra”

Compositions of the disclosure have several design features that provide superior properties. Porous silica optical tags can be organically functionalized, for example, with silane-based compounds. Moreover, the large surface area of the optical tags increases the probability of a binding reaction to a target molecule and increases the sensitivity of the detection of the target molecule. Using a light source of multiple wavelengths, spectroscopic analysis can reveal spectral features in the reflected or transmitted light, such as the wavelength, amplitude, phase, and/or number of spectral peaks or troughs. These spectral features contain encoded information useful to determine the identity of an optical tag. The encoded information can be used to identify which probes are bound to the optical tag, or for determining the identity of a target molecule that binds to or otherwise interacts with the optical tag.

Spectral peaks or troughs, useful for encoding information, can be created and controlled through alternating layers of fixed or varying porosity, such as a Bragg structure, through a single layer of continuously varying porosity, as in a rugate filter, or through various combinations thereof. A rugate is a gradient-index interference filter with a sine-wave refractive index profile. It is a type of index varying filter useful to provide an optical spectral code for the optical tags.

Porous silicon films have been shown to exhibit spectral properties dependent on thickness, porosity, and pore diameter. The pores are produced by means of an electrochemical etching wherein the etching current density determines the porosity, which is the volumetric fraction of the pores inside a layer of film, and the modulated structure of each pore in the film. Thus, in the case of rugate and/or Bragg filters, the etching current waveform that generates the pores in the porous silicon film determines spectral reflectance peaks or troughs. The film's porosity relates directly to the material's optical index of refraction. More porosity leads to a lower refractive index because the dielectric effective medium contains more air.

As a general overview, the optical tags can be encoded with unique signatures that can serve as a unique optical spectrum code for each tag or a component of the unique tag code, which are analogous to barcodes or digital fingerprints used to reference objects in a database. The unique signatures are associated with how each optical tag is configured to interact with electromagnetic waves. The optical tags can be scanned with an emitter of an electromagnetic wave or other source of electromagnetic waves. A receiver receives an energy spectrum (i.e., a “spectral response” or a “reflectance or transmission spectrum”), which is based on the interaction of the optical tag with the electromagnetic wave. In some instances, the emitter and receiver may be a single device collectively referred to as a “tag reader.”

The energy spectrum will have distinguishable spectral features. The characteristics of these one or more spectral features may be used to determine the unique spectral signature associated with the optical tag using various methods. Examples of the spectral features include spectral peak positions, spectral peak amplitudes, side lobes, and any other identifiable feature in the energy spectrum. Generally, these spectral features of the received spectral signature will be related to various configurable inputs used in manufacturing the optical tag. For example, the position and amplitude of a spectral peak may be associated with a sinusoidal component of an electrochemical etching waveform used to manufacture the optical tag, and the parameters of that waveform may be modified. Thus, the manufacturing process can configure optical tags to produce certain spectral features in the spectral signature. The combination of those spectral features may be used to determine the unique spectral signature associated with an optical tag, which may then be used to determine a unique optical spectrum code (i.e., a unique tag code) associated with the object.

This broad, but simplistic, overview of optical tags is provided out of convenience and overlooks many implementation details. For example, the optical tag may comprise a layered Bragg-like filter or a rugate filter. An optical interference filter may be a rugate filter made of porous silicon, porous silica, or varying proportions of both porous silicon and porous silica. More information on how silica micro-tags can be manufactured for use as a marker or identifier with an encoded unique signature, how a spectral signature of the optical tags can be analyzed to determine the unique spectral signature with a tag reader device, and additional optical tag implementation details are provided in U.S. Pat. No. 8,596,546; U.S. Pat. No. 8,881,972; U.S. Pat. No. 9,033,213; U.S. Pat. No. 8,453,929; U.S. Pat. No. 8,511,557, U.S. Pat. No. 9,523,634, U.S. Publication No. 2011/0304131, and U.S. Publication No. 2016/0123809. The aforementioned references are incorporated by reference in their entirety.

Porous silica or silicon optical tags (i.e., “microtaggants” or “tags” or “particles”) can be used for the detection of biological and chemical analytes. Silica can be organically functionalized, for example with silane-based compounds. The large surface area of the optical tags increases the probability of a binding reaction, and therefore increases sensitivity of the detection.

In various embodiments, optical tags comprise one of the following materials: silicon, fully- or partially oxidized silicon, silicon nitride, doped silicon, or any other appropriate material. Partially oxidized tags have a refractive index which is significantly higher than that of pure silica, which can facilitate an improved signal comprising a spectral signature. In some embodiments, optical tags comprise silica that is deemed “generally recognized as safe” (“GRAS”) by the U.S. Food and Drug Administration (FDA).

Each optical tag contains a designed spectral signature or set of spectral signatures chosen as a unique identifier. Optical tags with a given spectral signature are manufactured in quantities sufficient to enable cost-effective identification of analytes in commercial-scale product volumes (see, e.g., U.S. Publication No. 2011/0147456, “Labeling and Authenticating Using a Microtag,” incorporated by reference herein in its entirety). The number of available spectral signature combinations varies from hundreds to billions.

In one embodiment, the reflectance or transmission spectra of the optical tag is controlled to fall within a sufficiently narrow range of one or more bands of energy. In one embodiment, the reflectance or transmission spectra of the tags is measured and recorded, either for all tags or for a statistically meaningful subset of the population of tags.

If a silicon or silica porous thin film or fragment thereof with parallel top and bottom surfaces (e.g., an optical tag) is illuminated with light, certain wavelengths will be reflected preferentially. Fabry-Perot fringes result from the interaction of light with the abrupt interfaces at the top and bottom surfaces. Rugate reflections and Bragg reflections result from the interaction of light with the pores. In both case, the refractive index of the pore material and that of the surrounding material will affect the spectral position of the reflected peaks or troughs. This impact is a function of the encoded structure of the pores in each optical tag. Although provided herein as an example, optical tags are not limited to silicon-based materials. In some embodiments, an optical tag may be made from any porous material comprising pores that are both smaller than the wavelength of light and capable of generating a coded spectral response from an optical spectral code encoded in the optical tag. In some embodiments, an optical tag may be made from any porous material capable of generating a coded spectral response based on the index varying properties of the pores.

In some embodiments, the reflectance or transmission spectra from the optical tags comprises rugate spectral peaks or troughs. In some embodiments, the reflectance or transmission spectra from the optical tags comprises Fabry Perot peaks or troughs. In some embodiments, the reflectance or transmission spectra from the optical tags comprises both Fabry Perot and Rugate peaks or troughs. In some embodiments, the reflectance or transmission spectra from the optical tags comprises Bragg peaks or troughs. In some embodiments, the reflectance or transmission spectra from the optical tags comprises Bragg and Fabry Perot peaks or troughs. In some embodiments, the reflectance or transmission spectra from the optical tags comprises Bragg and Rugate peaks or troughs. In some embodiments, the reflectance or transmission spectra from the optical tags comprises Bragg, Rugate, and Fabry-Perot peaks or troughs. In some embodiments, the reflectance or transmission spectra comprises a distinguishable feature of a spectral signature that is a function of a the modulated pore structure of the optical tags.

Optical tags comprising modulated pore structure can be made by etching of a silicon wafer to form pores in the surface of a wafer. The etching can be performed using any suitable acid. Electrochemical etching can be performed such that a controlled spectral reflectance or transmission signature can be attained from the optical tags (see e.g., Meade et al., Porous silicon photonic crystals as encoded microcarriers. Adv. Mater., 2004. 16(20): p. 1811-1814). Recent technological development, e.g., by Trutag Technologies, has enabled significantly finer control of the reflected rugate peaks or troughs in a spectral signature with a high manufacturing efficiency for such tags (see, e.g., U.S. Publication No. 2011/0147456). This makes it possible to create a very large number of tag signatures in a controlled, repeatable and cost-effective way.

Once etched, a layer comprising etched pores is removed from the silicon wafer. The layer comprises etched pores with a modulated structure through the layer. Once formed, the layer of thin film can be broken into smaller fragments to form individual optical tags, each with a substantially similar pore structure. In some embodiments, the layer is fragmented by sonication, thereby forming porous silicon optical tags. The resultant optical tags contain a modulated porous structure that is programmed during the electrochemical etch to display a unique reflection or refraction (transmission) spectrum, i.e., a unique spectral signature. In some embodiments, the optical tags are then fully or partially oxidized.

In some embodiments, the spectral signature of an optical tag is measured via a spectrum-resolving reader to generate a reference spectral signature. In some embodiments, the reference spectral signature is verified against other information as part of a database, such as reference spectral signatures of other tags. In some embodiments, a set of reference spectral signatures observed from optical tags from the same layer or wafer or multiple layers or wafers can be analyzed to assess a statistical distribution of reference spectral signatures for the modulated pore parameters in the optical tag.

The optical tags provided herein can generate billions of possible distinct spectral signatures, using, e.g., peak number, peak placement (i.e., wavelength of peak), peak phase, and/or peak amplitude as modulation parameters. The optical tags are passive, inconspicuous and can be attached to the various substrates such as paper, plastic and glass. In accordance with one embodiment of the invention, a collection of optically-encoded silica tags is provided, each comprising a modulated porous structure encoded by the same or similar signal, thus generating a substantially similar spectral signature of a first type upon illumination.

Although embodiments of silica-based optical tags are taught, the optical tags can be generated using any material suitable to generate a spectral signature comprising a spectral response of an index varying filter, such as rugate or Bragg peaks or troughs. This can include any non-silica dielectric material with a refractive index sufficiently different from air to enable accurate detection and identification of a unique spectral signature.

In some embodiments, the reflectance and/or transmission spectra of these optical tags is controlled to fall within a sufficiently narrow range of one or more bands of energy. In some embodiments, the reflectance and/or transmission spectra of the generated optical tags is measured and recorded, either for all tags or for a statistically meaningful subset of the population of tags, in order to generate a statistically meaningful distribution of reflectance and/or transmission spectra from each unique porous structure encoded into the optical tags.

In a preferred embodiment, the optical tags are functionalized to facilitate binding of a probe to the optical tag. Preferably, functionalization occurs after the desired oxidation, if any is performed. In some embodiments, a linker, such as a silane linker, is bound to the optical tag. In some embodiments, the optical tag is bound to a linker at any point during the generation of the optical tag. For example, a silica wafer may be bound to a linker before the porous silicon thin film is removed from the wafer. A porous silica film removed from the wafer can be bound to a linker before it is sonicated to form individual optical tags. In some embodiments, a probe can be attached to the linker on a wafer prior to lift-off and sonication, or prior to sonication. A probe can also be attached to a linker of several individual optical tags from the same wafer (i.e., having a substantially similar modulated porous structure) after sonication.

In some embodiments, the optical tags comprising silica are functionalized with a thin layer of a linker, such as a silane, coated on the optical tags. Then, a specific probe is bound to the linker, thus functionalizing the optical tag with the probe. See, e.g., U.S. Pat. No. 4,034,072, incorporated herein by reference in its entirety, for an example of a binding of a probe to a silane linker.

In some embodiments of the invention, attachment of a target-specific probe to a functionalized optical tag comprises incubating a set of target-specific probe molecules with a solution comprising functionalized optical tags to allow binding of the target-specific probe molecules to the functionalized optical tags.

In one exemplary embodiment, the probe is an aptamer and is bound to a functionalized tag as follows: A 52 mM EDC solution is injected to a chamber containing the functionalized optical tags and allowed to react for 1 hour. Subsequently, 50 μL of 75 μM aptamer solution is applied to the optical tags for 1 hour, followed by thorough washing with 10 mL of 50 mM Tris buffer. This can be done through a filter with pores smaller than the optical tags. Finally, the aptamer is folded by incubation in PBS for 30 min.

In some embodiments, the probes are specific antigens. In some embodiments, the probes are enzymes. In some embodiments, the probes are antigen-associated antibodies. In some embodiments, the probes are oligonucleotides. In some embodiments, the probes are Peptide Nucleic Acids. In some embodiments, the probes are any molecule or optical tag with specific preferential bonding to a target analyte.

Probes comprising reactive moieties can be bound to the surface of a functionalized optical tag or within a pore of a functionalized optical tag, or a combination thereof. Thus, a reactive moiety can be present on the surface of an optical tag or in the pore of an optical tag, or both. In some embodiments, the reactive moiety is present only inside the pore of an optical tag (e.g., as part of a surface or volume within the pore).

Exemplary reactive moieties of the disclosure include, but are not limited to, at least a portion of a probe, such as a nucleic acid, an antibody, an aptamer, an antigen, an enzyme, a peptide nucleic acid, or any combination thereof. Exemplary target molecules of the disclosure include, but are not limited to, a nucleic acid, a protein, a carbohydrate, antibody, antigen, an inorganic molecule, an insecticide, a virus, a bacteria, a toxin, a hormone, or any combination thereof.

In certain embodiments, the reactive moiety of a probe may interact with the target molecule with high specificity, such as a high affinity binding reaction. The reactive moiety and the target moiety may interact via a covalent bond, such as a polar or nonpolar covalent bond, or via a non-covalent bond, such as an ionic bond, a polar bond, a hydrogen bond, a Van der Waals interaction.

In one embodiment, these optical tags are organically linked, for example via silanization, such that they are sufficiently uniformly coated yet the pore structure is not completely filled by the linker coating.

Without limiting the generality of the linking process, many methods for functionalizing silica surface are known to those familiar in the art. One is described herein as an example. A 1 mL aliquot of 0.5% 3-aminopropyltriethoxysilane (Aldrich Chemicals, Inc.) in ethanol is added to approximately 10⁵ optical tags and shaken for 1 h. The optical tags are washed with ethanol three times and once with acetonitrile in a centrifuge filter (Nanosep, 0.2 μm, Pall). Next, acetonitrile (980 μL), N,N-diisopropylethylamine (20 μL, Aldrich Chemicals, Inc.), and cyanuric chloride (10 mg, Aldrich Chemicals, Inc.) are added to the optical tags and the mixture is allowed to react for 2 h with constant shaking. The optical tags are then washed with acetonitrile 4 times, suspended in ethanol, and transferred to a microcentrifuge tube. The optical tags are settled by centrifugation at 4000 RPM and the supernatant decanted to a volume of approximately 25 μL.

In another embodiment, optical tag functionalization may be achieved as follows. The optical tags are incubated with a solution of 42 mM APTES in toluene for 1 h. After the solution is removed, e.g., via filtration of the optical tags, the optical tags are washed with toluene, ethanol, and acetone and dried under a nitrogen stream, for example in a chamber. The APTES-modified optical tags are then immersed in a freshly prepared solution of 100 mg of succinic acid in 4.7 mL of DMSO and 300 μL of 0.1 M NaHCO3, pH 9.4 for 30 min. After removal of the solution, e.g., by filtration of the optical tags, they are washed extensively with DMSO two times and with purified water.

Compositions and/or substrates of the disclosure may be heated or cooled relative to ambient or room temperature to facilitate contact and/or binding of a probe comprising a reactive moiety to the surface of an optical tag.

In one embodiment, one or more batches of optical tags with different spectral signatures of second, third, etc. types, are functionalized with specific probes, such that one probe species is exclusively associated with one or more spectral signatures (codes).

In another embodiment, one or more batches of optical tags with different spectral signatures are functionalized with specific probes, such that there may be some overlap between probe species and optical tag spectral signatures. By overlapping spectral signatures associated with a probe, one is still able to recover the identity of the bound target.

In one embodiment, functionalized optical tags with bound probes from different batches are mixed in powder form, e.g., when the tags are dry.

In another embodiment, functionalized tags with bound probes from different batches are mixed in a buffer solution.

Imaging

In some embodiments, the unique identifier, i.e., spectral signature, of each optical tag can be read by a spectral reader

Readout of optical tag spectral signature can be done by detecting a reflected or transmitted spectral signal from the optical tags. This has traditionally been done using refractive optics, grating or other spectrally dispersive mechanisms. These methods typically either analyze the spectrum from a spot or from a line. Therefore, in order to scan a region containing multiple porous silicon tags, the substrate can be scanned with respect to the light source and reflective probe, or vice versa. This typically results in an expensive system which includes moving stages, is less mechanical robust and therefore less amenable to being portable, is expensive, and requires a relatively long time to scan a large area with fine spatial resolution, which time is proportional to the ratio of total area to the required spatial resolution.

The development of tunable Fabry-Perot interferometers (FPI) in hyperspectral imaging is used by the methods of the disclosure to overcome many technical hurdles present when imaging distinct spectral signatures in a multiplexed reaction. FPI devices act as tunable filters with a wide spectral range and with fine spectral resolution. When placed in the illumination path between a white light source and a sample, a monochromator-type system may be constructed whereby one or a known number of narrow wavelength bands illuminate a target. When placed in the imaging arm of a system illuminating an object with white light, one or more wavelength bands of a transmitted optical signal and/or one or more wavelength reflection band(s) may be selected. When placed in the imaging arm of a system illuminating an object with white light, one or more of a known number of wavelength bands of a transmitted optical signal and/or one or more of a known number of narrow wavelength reflection band(s) may be selected. In certain embodiments, the one or more wavelength reflection band(s) may be narrow wavelength reflection band(s). In the latter case, the FPI may either be placed in the Fourier plane of the imager or on the focal plane. The transmitted optical signal and/or reflected image may be detected by an area sensor. Thus, a relatively wide area can be scanned relatively quickly, with a wide spectral range and fine spectral resolution. For example, hyperspectral imaging devices and methods of the disclosure may provide a 100 mm² field of view, a 400 nm scan range with less than 10 nm resolution and a total acquisition time of less than 5 seconds. (see, e.g., US Publication No 2016/0123809, “Fabry-Perot Spectral Image Measurement,” incorporate herein by reference in its entirety).

Substrates of the disclosure may be imaged by a hyperspectral imager with sufficient spectral resolution to decode one or more spectral signatures from the optical tags as well as spectral changes caused by binding of a target molecule with a reactive moiety on the optical tags. Thus, in certain embodiments, a single pixel of the image sensor on the hyperspectral imager may image, at most, a single optical tag.

Hyperspectral imagers of the disclosure may contain a tunable Fabry-Perot-Interferometer (FPI) whereby the FPI may be a component of either a monochromator-type instrument (i.e., the FPI selects narrow band(s) of illumination) or as a component of a spectrometer-type instrument (i.e., the FPI selects narrow band(s) of the transmitted optical signal and/or reflected signal to be imaged).

The spectra of all visible tags in the field of view may be concurrently collected via a reader with a tunable Fabry-Perot Interferometer (FPI). The location of individual optical tags may be determined by identifying unique characteristics of optical tag reflected signals versus reflected signals of the substrate (e.g. the existence of reflected spectral features). The location of individual optical tags may also be determined by identifying unique characteristics of optical tag transmitted optical signals versus transmitted optical signals of the substrate (e.g. the existence of transmitted optical spectral features).

In certain embodiments, a high numerical aperture of the illuminating light may be accomplished by using, for example, a large diameter lens or a diffuser and a low numerical aperture of the imaging optics may be accomplished using, for example, a pupil, such that a plurality of optical tags having various spatial orientations can be illuminated but only the specular reflections are collected/measured.

In certain embodiments, an illumination numerical aperture may be low, such that, for example, mostly collimated light illuminates the substrate, and a collection optics numerical aperture may be high, for example by using a large-diameter lens, such that specular reflections may be collected with high efficiency

While a reflected signal shows spectral features, a transmitted optical signal would show the inverse of the reflected spectral features. When reading signals from optical tags of the disclosure, the transmitted optical signals may appear to be the inverse of the reflected signal.

In certain embodiments, an optical tag provides rugate or Bragg spectral peaks or troughs. Here the term “filter” is used interchangeably to describe both a selective transmission and a selective reflection which are produced by a dielectric modulation, where the refractive index is varied continuously or in steps at least in some part of the structure. Rugate or Bragg fringes or peaks are distinct from Fabry-Perot fringes, which are produced from reflections from two or more parallel surfaces having abrupt transition in refractive index.

The simplest example is a structure with a sinusoidal oscillation of the refractive index, leading to reflection in some narrow wavelength region. In transmission, one obtains a notch filter, which blocks some limited wavelength range, while in reflection one obtains a bandpass filter. Such filters are used, for example, as laser blocking filters in Raman spectroscopy. It is also easily possible to combine multiple reflection bands in order to obtain multiple notch filters, for example.

There are different techniques for obtaining a continuous variation of the refractive index in a dielectric material. A common approach is to fabricate mixtures of two different materials with a variable mixing ratio. For that purpose, one may use double electron beam coevaporation or similar methods (also using resistance heating or ion beam sputtering) with material pairs such as Zr0₂/MgO, Zr0₂/Si0₂, Ta₂0₈/Ti0₂ or Ti0₂/Si0₂. Depending on the detailed growth conditions (composition, substrate temperature, etc.), polycrystalline or amorphous structures can result. For some coating materials such as Ti0₂, the packing density can be varied during vapor deposition, e.g. by control of the oxygen partial pressure or by glancing angle deposition. The packing density directly affects the refractive index. (Jerman et al., Refractive index of thin films of Si0₂, Zr0₂, and Hf0₂ as a function of the films' mass density, Appl. Opt. 44 (15), 3006 (2005)). Such techniques are also applied to porous silicon rugate filters. (Kaminska et al., Simulating structure and optical response of vacuum evaporated porous rugate filters, J. Appl. Phys. 95 (6), 3055 (2004)).

A challenge arises from the fact that a precise refractive index control is more difficult to obtain for index varying structures. For high precision, automatic computer control is required, based on online growth monitoring and a sophisticated algorithm. When deviations from the target values are detected during growth, the rest of the structure is automatically adapted such as to compensate the errors as far as possible.

In comparison with filters based on standard dielectric layers, rugate filters provide some special potentials. A sinusoidal oscillation of the refractive index can create an isolated peak in the reflectivity spectrum, without any significant sidebands as are obtained for ordinary Bragg mirrors. Such sidebands essentially arise from the higher-order Fourier components of a rectangular oscillation. However, a clean filter behavior of that type requires two additional measures: avoiding additional reflections from the ends and apodization. It is possible to linearly superimpose multiple oscillations of the refractive index in order to combine multiple reflection features. Rugate filters have also been reported to have substantially higher laser-induced damage thresholds, compared with conventional filters. (Jupe et al., Laser-induced damage in gradual index layers and Rugate filters, Proc. SPIE 6403, 640311 (2006)).

For a theoretical analysis, an index varying structure (e.g., a gradient-index coating structure) can be approximated by a step-index structure with a larger number of steps, such that the index change from one “layer” to the next one becomes very small. Therefore, software developed for step-index structures can be used. In some embodiments, the index varying structure is automatically computed from some set of parameters such as a medium refractive index, an oscillation amplitude, and parameters for apodization. In some embodiments, the rugate structure is combined with additional parts such as anti-reflection coatings.

Simple filter curves can be obtained with analytical designs. For more complex designs, one can use the inverse Fourier transform method, where one essentially exploits the fact that at least for low reflectivities, the reflection spectrum is related to the Fourier transform of the spatial index profile. The method can be modified to work also with high reflectivities. (Yerly et al., Synthesis of high rejection filters with the Fourier transform method, Appl. Opt. 28 (14), 2864 (1989)).

There are also other techniques for the numerical optimization of rugate or Bragg filters. For example, in some embodiments, the refractive index profile is optimized, such as by parameterizing the structure as explained above. One then can vary these parameters such as to minimize some kind of merit function, which “punishes” deviations from the desired optical properties.

Instead of looking at the spectrum as the reflected signal, the reflected signal may be any general optical signal correlated with the spectrum. For example it can be the digital numbers output by red, green and blue channels of an image sensor, which digital numbers are a linear combination of the intensities of the one or more orders passing through an interferometer such as an FPI for a given mirror gap.

In certain embodiments, imaging a substrate comprising immobilized optical tags may include identifying a spectral change associated with at least one spectral feature (i.e., a “spectral shift”). In certain embodiments, imaging a substrate comprising immobilized optical tags may include one of static imaging, stepper imaging, or scanning.

A reference hyperspectral image of a composition comprising a substrate and a plurality of optical tags may be recorded before the optical tags of the composition contact or bind a target molecule. The relative or absolute locations and select spectral features of the optical tags comprising reactive moieties bound to target molecules may be measured and recorded. To determine the presence of a spectral shift, a second sample hyperspectral image of the composition comprising a substrate and a plurality of optical tags may be captured after contacting a sample suspected of comprising a target entity. Here, a spectral shift would indicate a binding of a target entity to a reactive moiety bound to an optical tag. In some embodiments, a hyperspectral image of an optical tag is captured after a subsequent wash step.

Spectral shifts of the disclosure may include, by way of a non-limiting example, rugate reflections or Bragg reflections. Spectral shifts of the disclosure may include, by way of a non-limiting example, Fabry-Perot fringes. Fabry-Perot fringes result from reflected light between two planes of an optical tag. For example, in certain embodiments, optical tags of the disclosure may comprise at least two flat surfaces that are parallel to one another and reflections between these at least two flat surfaces may be Fabry-Perot fringes.

Substrates

Substrates of the disclosure may comprise any material that can withstand the methods of the disclosure, including any solvents used, without degradation. Exemplary substrates of the disclosure include, but are not limited to, glass, paper, plastic, a polymer, or any combination thereof. As one example, the substrate can be a test strip or other device for receiving a sample. Substrates of the disclosure may further comprise a coating material suitable for immobilizing the plurality of optical tags. Exemplary coating materials include, but are not limited to, a glue, adhesive and a resin or a molecule with affinity to the optical tag material to which optical tags of the disclosure attach. Optionally, the substrate comprising a coating material such as a resin and in contact with a plurality of optical tags may be cured to immobilize the optical tags within the resin or the substrate.

Substrates may comprise one or more fluidic channels forming at least one inlet and at least one outlet. In a preferred embodiment, at least one surface of the one or more fluidic channels is optically visible/detectable in the spectral band of illumination and imaging. In some embodiments, one or more fluidic channels leads to a chamber or other device that provides a mechanism to detect the spectral band of illumination and imaging of the optical tags.

Substrates can include fluidic actuation elements such as electrodes (in the context of a digital fluidics system), membranes (in the case of pressure-induced fluidics actuation), etc. In some embodiments, substrates comprise electrodes operably connected to assist in increasing the concentration of target molecules at the porous surface and/or to assist in removing non-specifically bound material from the pores upon completion of target incubation in the pores.

Substrates that comprise a coating material can contact the coating material by submersion of the substrate in the coating material or infusion of the substrate with the coating material. In some aspects of this embodiment, the substrate is porous and, furthermore, may contain one or more fluidic channels.

In some embodiments, substrates comprise one or more topographic features that physically capture a plurality of optical tags upon contact. For example, one or more topographic features of the substrate may include an arrangement of concave or convex impressions and/or protrusions that may be oriented randomly or in a pattern. For example, the substrate may include an arrangement of concave or convex impressions and/or protrusions (e.g., pits or grooves) that are printed, embedded or etched onto the substrate. The substrate may include a lithographically-defined or otherwise fabricated to comprise an arrangement of adhesive features that may be concave or convex impressions and/or protrusions. The substrate may include fibers which are used to non-specifically capture silica optical tags.

In some embodiments, the optical tags are dried and deposited on a substrate such that they are immobilized on the substrate at a preferential orientation. In some embodiments, the optical tags are immobilized such that parallel faces of a silica optical tag are largely parallel to the surface of the substrate. In some embodiments, optical tags immobilized to the surface of the substrate are oriented randomly with respect to the surface of the substrate.

In some embodiments, deposition of the optical tags on the surface of a substrate may be achieved by spray. In some embodiments, optical tags may be embedded in a solvent and then sprayed on a substrate, and the solvent may be wholly or partially disintegrated, for example by evaporation, such that the top surfaces of the optical tags are exposed. In some embodiments, the optical tags may be flowed over a substrate, either via a nozzle or through a fluidic channel and allowed to settle or bond to the substrate, such that the top surfaces of the optical tags are exposed.

In some embodiments, the substrate comprises a sticky substance, such as a glue or resin, which facilitates immobilization of the optical tags to the substrate. In some embodiments, the substance is subsequently cured to affix the tags to the substrate. In some embodiments, electromagnetic radiation may be used to immobilize the optical tags on the substrate, e.g., by initiating cross-linking of an adhesive.

In some embodiments, optical tags are immobilized to the surface of a substrate by laminating the substrate comprising optical tags on the surface with a porous material comprising pores smaller than the size of a significant percentage of the tags but larger than the size of the target molecules.

In some embodiments, a mechanical force is applied to align the surfaces of the optical tags parallel to the surface of the substrate. In some embodiments, the mechanical force is provided by a comb, a laminate, a roller or a sufficiently flat object pulled parallel to the surface of the substrate. In some embodiments, the optical tags may be physically captured on a substrate, for example between fibers or within topographical features present or formed on the substrate. In some embodiments, the coating or adhesive used to bind the particles to the substrate provide an attractive force normal to the substrate surface to orient the flat surface of the optical tags in a configuration that is parallel to the surface of the substrate.

Compositions and/or coating materials of the disclosure may be heated or cooled relative to ambient or room temperature to facilitate contact, immobilization, or orientation of the plurality of optical tags to a surface of the substrate.

In some embodiments, multiple unique optical tags each encoded to generate a unique spectral signature (i.e., a known optical spectral code associated with one or more specific attributes of a reflectance or transmission spectrum) are applied to a substrate. Each unique spectral signature is associated with at least one probe and target entity. In some embodiments, the number of tags of each type is inversely proportional to the relative sensitivity of the probe in that tag to its target. In other words, the higher the affinity of a certain probe to its target, the fewer tags containing that probe will be incorporate onto a substrate. And similarly, the higher the required sensitivity of a certain probe to its target, for example, because the target is expected to be found in lower concentrations, or has a lower binding affinity, the more tags containing that probe will be incorporated on the substrate.

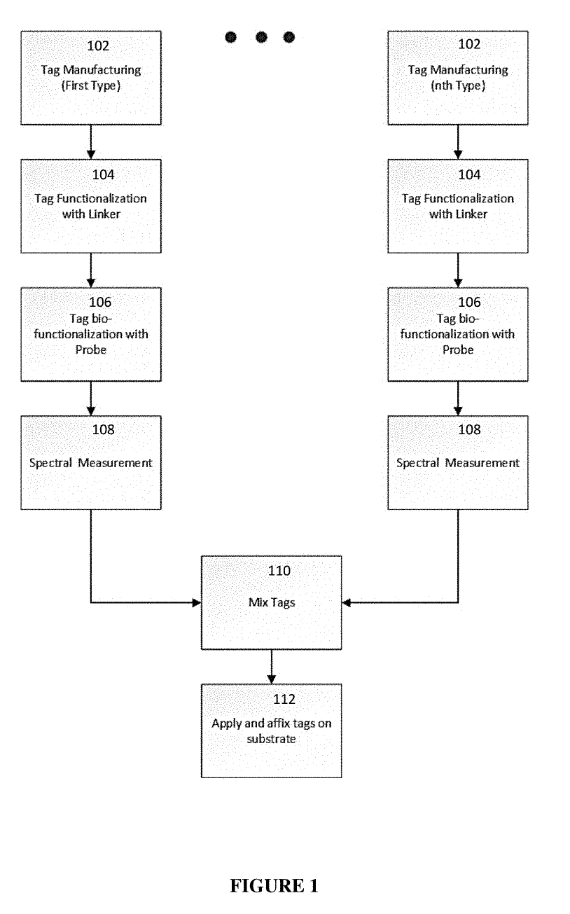

FIG. 1 is a flow diagram providing a method for generating unique functionalized optical tags and applying them to a substrate, according to an embodiment of the invention. It should be understood that the steps of the flow diagrams provided herein are illustrative only. Different embodiments of the invention may perform the illustrated steps in different orders, omit certain steps, and/or perform additional steps not shown in the flow diagrams. The method can start and end at various points in the process, and typically is a continuous process with multiple steps occurring simultaneously. So the flow diagrams, including FIG. 1, provide only an example of one ordering of method steps.

In the method of generating a substrate with a plurality of unique functionalized tags, the process includes generation of two or more unique functionalized optical tags each capable of generating a unique spectral signature each associated with one or more probes, reactive moieties, or target entities. A set of tags with a unique optical spectral code are manufactured 102, e.g., by etching a modulated pore structure into a wafer, removing a layer comprising the wafer, and breaking the layer to form multiple optical tags. The set of unique optical tags is then functionalized with a linker 104, and bound to a probe specific for a target analyte 106. The probe is correlated with the unique optical spectral code for the set of optical tags. A spectral measurement of one or more tags in the unique set of optical tags can be taken to determine a reference spectral signature 108. The process of manufacturing 102, functionalization 104, probe binding 106, and spectral signature measurement 108 can be repeated n times for each of n sets of optical tags with unique optical spectral codes. The n sets of unique optical tags can then be added to a single mixture 110 and immobilized onto a substrate 112. Thus, a substrate capable of detecting multiple target analytes is generated, where the spectral signature from each optical tag can determine whether a target entity has reacted with a probe and the identity of the target entity from a plurality of possible target entities.

Substrates provided herein may comprise a plurality of optical tags immobilized to the surface, including, for example, at least 5, at least 10, at least 20, at least 50, at least 100, at least 500, at least 1,000, at least 10,000, or at least 100,000 optical tags. In some embodiments, the substrate comprises from 100 to 1,000 optical tags on the surface of the tag.

Compositions of the disclosure may include at least 10 unique optical tags immobilized to the surface of a substrate, each comprising a unique probe capable of reacting with a unique target entity from a set of at least 10 target entities, wherein the at least 10 optical tags each produce a unique spectral signature when illuminated. Compositions of the disclosure may include at least 50 unique optical tags immobilized to the surface of a substrate, each comprising a unique probe capable of reacting with a unique target entity from a set of at least 50 target entities, wherein the at least 50 optical tags each produce a unique spectral signature when illuminated. Compositions of the disclosure may include at least 100 unique optical tags immobilized to the surface of a substrate, each comprising a unique probe capable of reacting with a unique target entity from a set of at least 100 target entities, wherein the at least 100 optical tags each produce a unique spectral signature when illuminated. In some embodiments, the different reactive moiety and different spectral signature permit the identification of at least two different target molecules. In some embodiments, each optical tag having the same binding moiety or probe has the same spectral signature.

In some embodiments, multiple unique optical tags may be used to detect a single target, for example, whereby multiple probes specific to different regions of the targets may be incorporated on multiple respective tags, each containing a unique encoded spectral response.

The disclosure provides a method of obtaining a signal from a highly multiplexed, label-free assay, comprising contacting a substrate comprising a plurality of functionalized optical tags with a sample, the plurality of optical tags each bound to a probe with chemical or biochemical specificity for a target; incubating the sample to a predetermined percentage of completion of a reaction, such as a binding reaction or an enzymatic reaction; and imaging the substrate to obtain an output signal associated with at least one of the plurality of probes or targets. In some embodiments, the imaging comprises collecting a reflected signal or a transmitted signal.

In some embodiments, a substrate comprising functionalized optical tags is lyophilized and or otherwise packaged dry to facilitate long term storage. The substrate can be can be reconstituted before use. In another embodiment, a substrate comprising functionalized optical tags is packaged in a buffer solution such that the functional probes are minimally degraded over time.

Method of Detection

Compositions comprising a plurality of optical tags may be flowed across a surface or through a fluidic channel of a substrate. Upon contact, the plurality of optical tags may be allowed to contact or settle onto the substrate via gravity. Alternatively, upon contact, the plurality of optical tags may be guided into contacting the substrate via a direct or alternating electric, magnetic or electromagnetic field.

In some embodiments, the substrate or an optical tag may contact a solution containing the sample to be interrogated. In other embodiments, the substrate or an optical tag may be exposed to a gaseous or vapor phase comprising the sample to be interrogated.

In some embodiments, functionalized optical tags or substrates comprising functionalized optical tags are heated or cooled relative to ambient or room temperature to facilitate binding or other interactions of a target entity with a reactive moiety of a probe. In some embodiments, functionalized optical tags or substrates comprising functionalized optical tags are physically or mechanically disturbed (e.g., by vibration) to facilitate binding or other interactions of a target entity with a reactive moiety of a probe.

In some embodiments, a sample is applied to a substrate comprising a plurality of functionalized optical tags on the surface of the substrate, and any unbound material is subsequently washed away. In a specific embodiment, a substrate with aptamer-functionalized tags is incubated with a target protein solution (in PBS-T) for 1 hour. After removal of the protein solution and washing the substrate with PBS, the sample may be incubated for 30 min in PBS before detection of a spectral signature from each of the optical tags to determine binding of the target protein to the aptamer bound to the optical tag.

In another embodiment, a plurality of unique functionalized optical tags are incubated with the target in solution and then filtered through a filter with pores smaller than the size of a majority of the tags. The filter is then dried and optical tags on the filter are imaged and analyzed using a hyperspectral reader as described herein.

In some embodiments, the functionalized optical tags can be exposed to a sample in vapor or gas form, then applied to a substrate for detection of a spectral signature to indicate whether a target molecule was present in the vapor or gas.

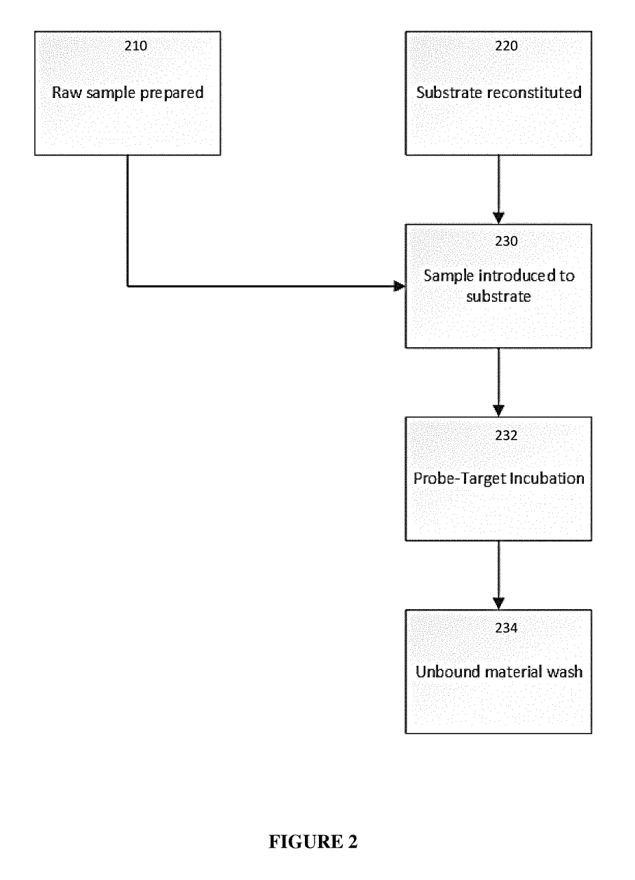

FIG. 2 is a flow diagram providing a method for a analyte binding to a substrate comprising optical tags. A sample can be collected and optionally undergo selected reactions to increase the detection of analytes therein, including filtration, isolation, or amplification of analytes 210. If the substrate has been used previously, it can be reconstituted 220. When both substrate and sample are ready, the substrate contacts the sample 230. Binding of an analyte to probes bound to optical tags on the substrate may be facilitated through incubation of the sample with the substrate 232. Before detection, the substrate can then be washed to remove unbound or weakly bound molecules from the optical tags 234.

In some embodiments, the imaging comprises identifying a spectral shift associated with at least one spectral reference feature in a spectral signature. In some embodiments, the imaging comprises one of static imaging, stepper imaging, or scanning. In some embodiments, before the receiving, the method comprises identifying an optical signal from the solid surface. In embodiments, the method comprises comparing the optical signal identified from the solid surface with the output signal associated with the at least one probe. In embodiments, the imaging comprises collecting a reflected signal or a transmitted signal. In some embodiments, imaging refers to collection of a hyperspectral image.

In some embodiments, the substrate is imaged by a hyperspectral imager with sufficient spectral resolution to decode the optical spectral codes from the tags as well as spectral shifts caused by binding of a target with a probe, such that in high likelihood, a single pixel of the image sensor on the hyperspectral imager will image at most a single tag. In some embodiments, the hyperspectral imager contains a tunable Fabry-Perot Interferometer (FPI) (see, e.g., U.S. Publication No. 2016/0123809, “Fabry-Perot Spectral Image Measurement,” incorporated by reference herein in its entirety) whereby the FPI is used to form either a monochromator-type instrument (whereby the FPI selects narrow band or bands of illumination) or to forma spectrometer-type instrument (whereby the FPI selects narrow band of bands of the reflected signal to be imaged).

In some embodiments, the numerical aperture of the illuminating light is set to be high, for example by using a large diameter lens or a diffuser, and the imaging numerical aperture of the imaging optics is low, for example by using a pupil, such that a large population of tags of various spatial orientations can be illuminated but only the specular reflections are collected. In other embodiments, the illumination numerical aperture is low, such that, for example, mostly collimated light illuminates the substrate, and the collection optics numerical aperture is high, for example by using a large-diameter lens, so that specular reflections may be collected with high efficiency.

In some embodiments, the spectra of all visible tags in the field of view is concurrently collected via a reader with a tunable FPI. In an embodiment, the location of individual tags is determined by identifying unique characteristics of tag reflected signals versus that of the substrate, such as the existence of reflected spectral peaks or troughs. In another embodiment, the tags are imaged with a non-FPI imager, which imager can resolve sufficient spatial and spectral features from the substrate containing the tags as to resolve individual tags and infer their optical spectral code.

In some embodiments, imaging a substrate comprising functionalized optical tags may include one of a direct or indirect reaction monitoring. For example, in the case of direct reaction monitoring, the position of certain spectral features of the optical tags on a substrate prior to the reaction taking place is either known or is measured. While the sample has been flowed onto the substrate and the target molecules react with the optical tags on the substrate, a spectral signal is recorded from the various optical tags. The difference between the signal from any optical tag during the incubation phase and the baseline signal from the same optical tag is a function of the refractive index shift versus baseline. This shift results from infiltration of the buffer solution into the pores, from diffusion of non-specific molecules into the pores, and from specific binding of target molecules onto the pore surface. The first two processes are static over time, resulting in a constant spectral shift with a Poisson noise. The last process is time-varying. By monitoring the reflectance spectrum in situ during the incubation we can use the different time scales of each process to separate their effects. This process can be detected either directly via the refractive index shift due to the incorporation of the target molecules or indirectly, for example if the incorporation of the target molecule on the surface of the pores induces a change in the hydrophobicity of the surface thus either forcing water out or letting water molecules into the pore and thus generating an amplified optical signal.

In some embodiments, a reference hyperspectral image of the tagged substrate is recorded before the introduction of the sample to the optical tag. For embodiments where the optical tag is immobilized on the surface of a substrate before contacting with a sample, a relative or absolute locations and select spectral features of a reference spectral signature of each optical tag can be determined and saved. Features of the reference spectral signature from each optical tag can be compared with a spectral signature obtained from the optical tag after contacting the sample suspected of comprising a target molecule. This comparison can determine whether a spectral shift, indicative of the binding of a target molecule to a reactive moiety of a probe bound to the optical tag, is present. The reference spectral signature associated with at least one optical tag or optical tag bound to a reactive moiety, whether bound to a substrate or not, may be previously determined in a separate experiment or provided through, for example, a database.

In some embodiments a hyperspectral image of an optical tag immobilized on the surface of a substrate is captured after contacting the sample suspected of comprising a target molecule, and, optionally after a wash step. A software program can be used to match spectral signatures from optical tags on the substrate with the reference spectral signatures for each optical tag, and to compare the spectral features of the reference and sample spectral signatures to determine whether there has been a spectral shift. The program can annotate those optical tags which display a spectral shift greater than a pre-determined threshold, which threshold is greater than standard measurement noise and the shifts resulting from non-specific molecular interaction. The program can also assess the shifts of many tags in aggregate, and based on the shift statistics gathered report: (1) a likely concentration of the target analyte, and (2) an estimated probability associated with the result. The program can then decode the optical spectral code in the shifted spectra tags, for example using methods described in U.S. Publication No. 2011/0147456 and U.S. Publication No. 2016/0123809.

Target molecules may bind to more than one optical tag, each having a distinct reactive moiety, in a multiplex reaction. In some embodiments, a software program may be used to determine the identity of the target molecule when a spectral shift has been detected in a specified plurality of optical tags in the multiplex reaction.

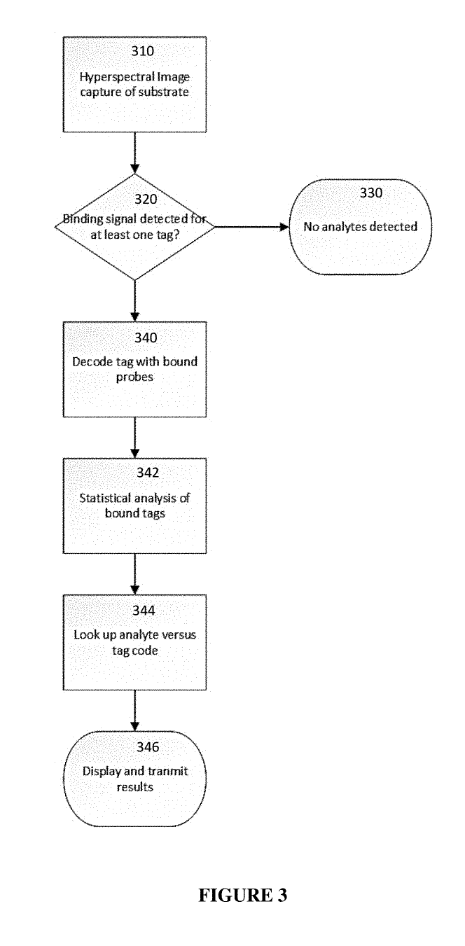

FIG. 3 is a flow diagram providing a method for sample analysis flow using an observed spectral reference from the optical tag. A sample spectral signature of individual optical tags exposed to a sample is captured using an imager 310. The sample spectral signature is used to determine whether or not there has been a binding interaction with a probe bound to the optical tag 320. If no shift in spectral signature is detected, that indicates that no binding interaction occurred 330. If a shift in spectral signature is detected 340, a statistical analysis of the bound tags can be performed 342, and the analyte identity determined by linking the spectral code to a unique spectral code of an optical tag 344. The results can be displayed and/or transmitted to another entity 346.

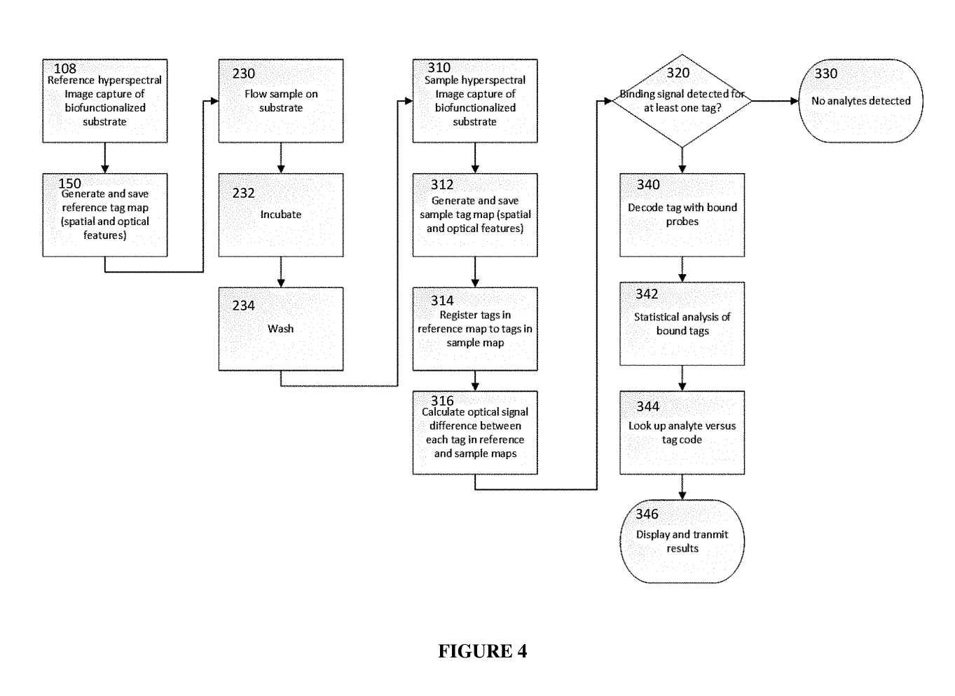

FIG. 4 is a flow diagram providing a method for detecting a target analyte by comparing a sample spectral signature with a reference hyperspectral signature taken before contacting the optical tag with the sample, and transmission of results, according to an embodiment of the invention. Here, a reference spectral signature is taken from each optical tag after functionalization and binding to the probe 108. The reference spectral signature is processed to generate a reference tag map with, e.g., spatial and optical features of the reference spectral signature, and saved to a memory 150. A sample is prepared and flowed on the substrate 230. Binding of an analyte to probes bound to optical tags on the substrate may be facilitated through incubation of the sample with the substrate 232. Before detection, the substrate can then be washed to remove unbound or weakly bound molecules from the optical tags 234. A sample spectral signature of individual optical tags exposed to a sample is captured using an imager 310. The sample spectral signature is processed to generate a sample tag map with, e.g., spatial and optical features of the sample spectral signature correlated with the reference spectral signature tag map 312. The reference and sample tag map are compared 314 to determine if there has been a shift in the spectral signature from an optical tag 316. A determination is then made as to whether or not there has been a binding interaction with a probe bound to the optical tag 320. If no shift in spectral signature is detected, that indicates that no binding interaction occurred 330. If a shift in spectral signature is detected 340, a statistical analysis of the bound tags can be performed 342, and the analyte identity determined by linking the spectral code to a unique spectral code of an optical tag 344. The results can be displayed and/or transmitted to another entity 346.

In some embodiments, a known manufacturing specification is used as a reference for comparison to a sample spectrum signature. A sample hyperspectral image is collected as described herein and spectral features are compared with a known manufacturing specification of the functionalized tags. A software program annotates those tags which display a spectral shift greater than a pre-determined threshold versus the manufacturing specification, which threshold is greater than standard measurement noise and the shifts resulting from non-specific molecular interaction. The program then decodes the optical spectral code in the shifted spectra tags. In one embodiment the identity of the target or targets corresponding to the shifted tags' optical spectral codes is looked up in a software lookup table.

A software program can be used to determine a binding of an analyte from the sample spectral signature based on a statistical analysis of the number of tags of each species which display a spectral shift. The statistical analysis can be based on a priori information regarding the sensitivity and specificity of each probe to its target in the presence of other analytes.

In some embodiments, a software program determines a binding of an analyte based on a statistical analysis of the number of optical tags having each reactive moiety that display a spectral change. This statistical analysis may be based on a priori information regarding the sensitivity and specificity of each reactive moiety to its target analyte in the presence of other pairs of reactive moieties and target molecules.

A multiplex reaction chamber comprising multiple optical tags with distinct reactive moieties may be incubated with one or more target The substrate may be dried and imaged using a hyperspectral imager or any other sensing element able to detect and decode the encoded information as well as optical changes resulting from an incorporation of a target molecule. Spectral readers of the disclosure can resolve sufficient spatial and spectral features from a substrate comprising optical tags of the disclosure to resolve individual optical tags and identify their spectral signature(s).

A combination of the spectral shift measured in the optical tags and the number of optical tags in which the spectral shift is observed may be used along with binding sensitivity and optical tag number information to estimate the quantity of the target molecule detected.

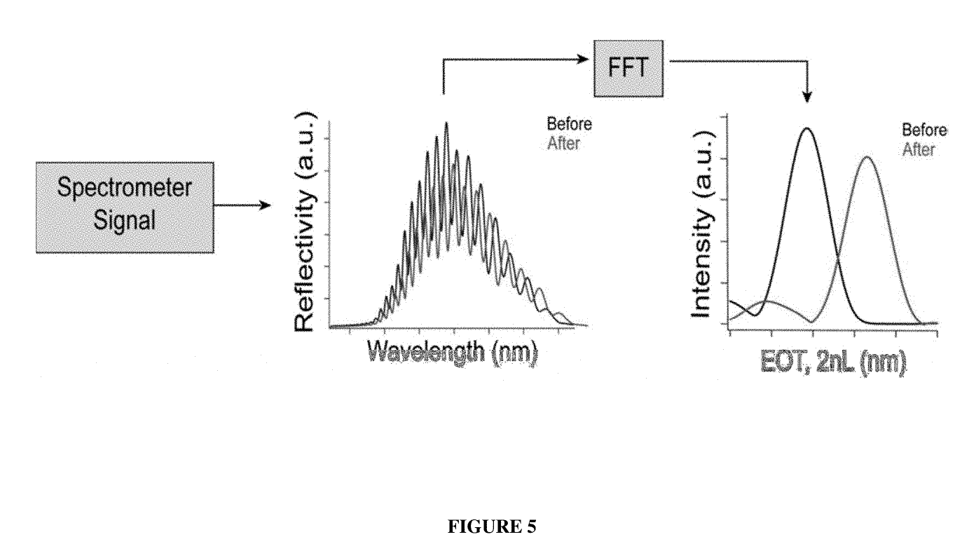

FIG. 5 is a graph illustrating an optical shift due to binding of a target with an optical tag, according to an embodiment of the invention. Here, an interference spectrum from an optical tag is detected by an interferometer at two different possible states (e.g., before and after exposure to a sample comprising an analyte). The interference spectrum at each state is then converted to an effective optical thickness at each state by applying fast Fourier transform (FFT) to the interference spectrum.

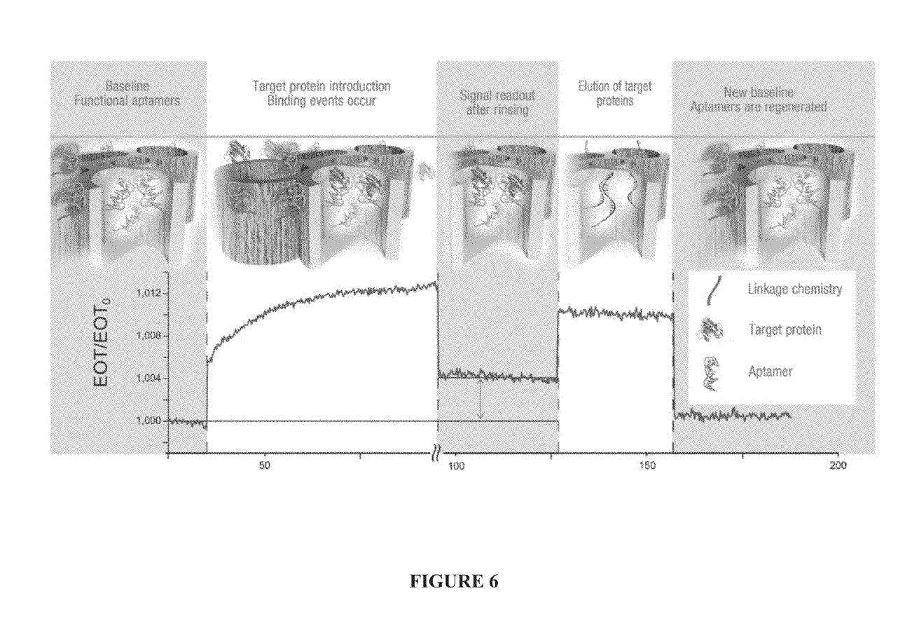

FIG. 6 is a schematic diagram depicting exemplary signals generated before, during and after a binding event of a target molecule to a reactive moiety attached via a coating material to a porous silica substrate. FIG. 6 shows a change in effective optical thickness (EOT) of an optical tag as compared to an initial reference point (EOT₀) over time during several states. A functionalized optical tag is exposed to a sample comprising a target analyte (e.g., a target protein) that binds specifically to the probes (e.g., aptamers) attached to the optical tag. When the analyte binds to the probes, EOT/EOT₀ increases as more binding events occur. After removal of non-specific binding by rinsing the optical tag, EOT/EOT₀ reflects the target analytes bound to the optical tag. After the assay, the proteins can be eluted and the optical tag reconstituted for reuse.

In the case of indirect reaction monitoring, optical tags may have openings that are contacted with, for example, a coating material comprising a protein, such that the opening on the top and bottom surfaces of the optical tag are blocked. When a sample, e.g., an enzyme, specific to that particular protein is contacted to the optical tags, the enzyme will remove the protein coating, allowing the pore to be filled with liquid, resulting, for example, in significant amplification of the optical shift. When the amplified spectral shift is detected, the particular optical tag can be identified by its spectral signature. Accordingly, the target molecule associated with the particular optical tag can be identified.

A blocking layer may be deposited over the substrate such that it also covers the plurality of optical tags. The blocking layer may either cover the pores or may fill the pores of the optical tag. Optical tags may be covered with a blocking layer on at least two flat surfaces that are parallel to one another. The blocking layer may comprise a protein. Target molecules of the disclosure may selectively remove the blocking layer. For example, a sample may contain one or more enzymes (e.g., proteases) and the blocking material covering at least one type of optical tag may contain reactive moieties sensitive to these proteases. Upon contacting the target molecule to the reactive moiety of the optical tag, the proteins blocking (covering or filling) the pores may be dissolved, resulting in a refractive index change (i.e., a spectral signature) of the optical tags.

A multiplex reaction having multiple optical tags with distinct reactive moieties may be incubated with one or more target molecules. Optical tags may be filtered through a filter with pores smaller than the size of a majority of the optical tags. The filter is then dried and imaged using a hyperspectral reader or a non-FPI imager. Spectral readers and imagers of the disclosure can resolve sufficient spatial and spectral features from the substrate containing the optical tags to resolve individual tags and identify/distinguish their distinct spectral signatures.

Pore dimensions may be used for size selecting which molecules can enter the pores. In a chemically-noisy environment such as bodily fluids, a large population of molecules is present. Non-specific signals due to infiltration of the pores by non-target molecules can be significantly reduced using the methods described above. Additional reduction can be attained by etching the pores such that they exclude molecules larger than the target molecules.

Samples comprising target molecules of the disclosure may be prepared by any means to increase access of the compositions and optical tags of the disclosure to the target molecules. For example, a sample to be analyzed may be treated to release the target analytes into a solution by physical or chemical cell lysis. A raw sample may be dissolved, purified, filtered, isolated, minced, or otherwise processed so that it can be flowed above (with or without a buffer) and bind efficiently with a reactive moiety of an optical tag of the disclosure. In some embodiments, target analytes or probes of the disclosure may be activated to facilitate access and/or binding between reactive moiety and a target molecule, for example, by removing a chemical blocking group from the probe or target molecule. In some embodiments, the sample undergoes a chemical amplification step to increase the concentration of analytes in a sample, such as PCR, isothermal PCR, TwistDX (enzyme-assisted accelerated amplification).

In some embodiments, the method may further comprise, before the post-incubation imaging, washing the substrate to remove analytes from the sample that are non-specifically bound to the substrate or optical tag. In some embodiments, washing may occur via blowing air, using a vacuum, creating a Venturi effect by blowing air over the pores, using mechanical agitation such as sonication, or using an electric field.

Substrates may be reconstituted by removal of the plurality of optical tags and/or coating materials (such as linkers) or target molecules from the optical tags or the substrate, and optionally, cleaning the substrate, followed by a redeposition of a coating material and new optical tags.

A large variety of samples are suitable for use with the compositions, systems and methods for detection provided herein.

In some embodiments, the sample contains dissolved powders such as milk, baby formula or instant coffee. Without loss of generality, target analytes may include melanin, insecticides or other relevant materials, and the probes may be molecules with sufficient specificity and selectivity to preferentially bind to said analytes. In some embodiments, the sample may contain milk or dairy products, or eggs or egg products, and, without loss of generality, the analytes may include hormones, whereas the probes may be molecules with sufficient specificity and selectivity to preferentially bind to said analytes.

In some embodiments the sample may contain either raw or filtered juice, or food processed to a liquefied solution, and, without loss of generality, the analytes may include insecticide molecules of interest, whereas the probes may be molecules with sufficient specificity and selectivity to preferentially bind to said analytes. The analytes may include toxins common in food poisoning, whereas the probes may be molecules with sufficient specificity and selectivity to preferentially bind to said analytes.

Samples may contain bodily fluids from a human or non-human species. Samples may include target molecules including, but not limited to, toxic and/or infectious materials (including viruses) as well as contaminants. Samples can contain one or more target entities including, but not limited to, toxic and/or infectious materials, bodily fluids from human and/or non-human species, viruses, or bacteria.

In some embodiments, the sample may contain oligonucleotide molecules, and the probes may be other oligonucleotides or proteins with sufficient specificity and selectivity to preferentially bind to said oligonucleotides or to sufficiently change said protein's structure as to result in a sufficiently large deviation in refractive index of the tag such that it can be detected as described herein.

In one embodiment, the sample may be a liquid, such as water, and the analytes may be potential contaminants, whereas the probes may be molecules with sufficient specificity and selectivity to preferentially bind to said analytes.

Results Analysis and Information Storage

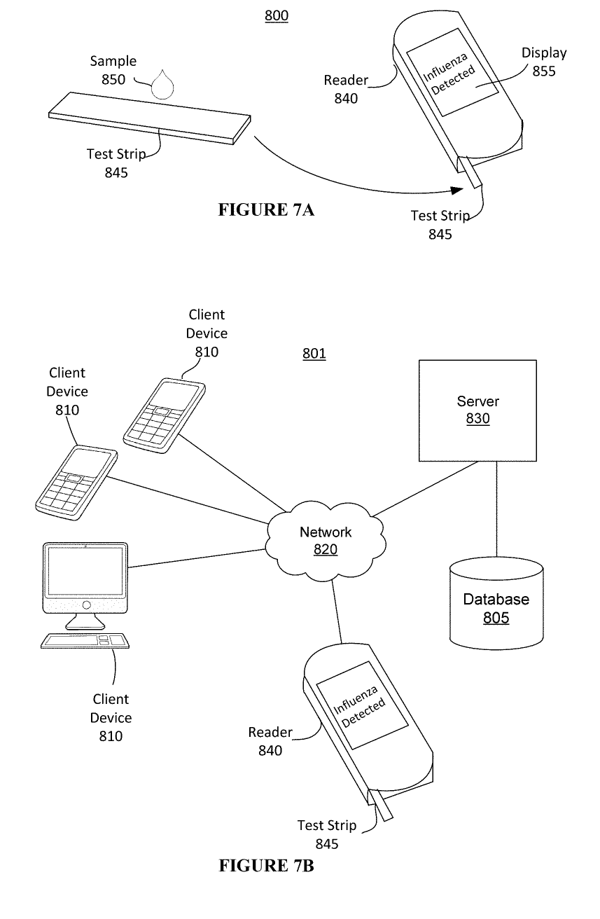

In some embodiments, the functionalized optical tags are included as part of a system, such as a diagnostic system or platform that provides highly-multiplexed consumer diagnostic results to users. Such a system can be used to detect various different analytes, including pathogens and biomarkers. It can be a low cost system designed such that a single test can replace a large number of different discreet laboratory tests. FIG. 7A illustrates an example of a diagnostic system 800 in which a sample 850 is added to a substrate, such as a test strip 845.

The test strip in this example can be prepared using a library of functionalized optical tags (e.g., porous silicon) deposited on plastic, paper, glass, or another material. In some embodiments, the optical tag functionalization occurs in large batches (e.g., hundreds of millions of tags at a time). Various methods can also be used in applying the optical tags to the test strip, including any of the methods described previously. In one embodiment, a powder is formed of batch functionalized tags with a specific coating and a target. This process is repeated for other target batches, each with its own tag signature but similar reference energy. In some embodiments, equal or known quantities of tags from all batches are mixed. The substrate strips (e.g., test strip 845) can be coated with an adhesive surface or other material with adhesive properties and then sprayed with the tag powder. The test strips 845 can then be packaged and provided to users for use in the diagnostic platform.

Users can apply the sample 850 to the test strip in various manners, such as by licking the strip, spitting onto the strip. Pipetting sample on to the strip, delivering sample via capillary action, amongst other options. In this example, the sample may be a body fluid, such as saliva or blood. In some embodiments, there is a wash step following the application of the sample to the strip. In some embodiments, various other sample preparation steps occur, such as a cell lysis step.

Reader 840 is used to read the test strip 845. For example, the test strip 845 may be inserted into the reader 840, placed on or inside the reader, scanned by the reader on a surface, or otherwise read by the reader 840. In some embodiments, the reader 840 is a diagnostic device or instrument. In one embodiment, the reader 840 is a compact hyperspectral imaging device that simultaneously images and decodes multiple tags (e.g., hundreds or thousands of tags) in a large field of view. The reader 840 identifies tags whose spectrum has shifted due to a binding event. In other words, binding events of label-free analytes can be optically detected as spectral shifts of tag reflectance peaks or troughs. The reader 840 then reads the optical spectral code associated with the tags, and determines the identity of the analyte. For example, the system can detect a pathogen, such as an influenza pathogen. In some embodiments, the reader 840 includes a display 855, such as a screen, that allows it to display to the user the results of the test. In some embodiments, the confidence level of the detection can also be determined and provided for display on the reader 840.

In some embodiments, a software program installed on the reader 840 may compare one or more spectral signature(s) from optical tags in a reference image. The program may then identify those optical tags that demonstrate a spectral shift greater than a pre-determined threshold. The threshold may be set to distinguish one or more spectral shifts from standard measurement noise and/or one or more spectral shifts resulting from a non-specific molecular interaction. The program then decodes the optical spectral code in the shifted spectra optical tags. In some embodiments, the software program is a computer readable medium storing instructions on the reader 840 that when executed by a processor within the reader 840 cause the processor to perform certain actions, such as reading the test strip or storing certain data. In some embodiments, the software program comprises one or more software modules that perform each of the various functions described above for the reader.

FIG. 7B illustrates a system environment 801 including the diagnostic system 800 of FIG. 7A, according to an embodiment. The system environment 801 further includes one or more client devices 110, one or more servers 130, a database 805 accessible to the server 130, where all of these parties are connected through a network 820. In other embodiments, different and/or additional entities can be included in the system environment 801.

The system environment 801 allows the results from the reader 840 to be shared via network 120 with one or more other users at their client devices 110, including being shared with family, friends, physicians or other medical personnel, schools, civil response teams, among others. Results can also be uploaded to the web.

The network 820 facilitates communications between the components of the system environment 801. The network 820 may be any wired or wireless local area network (LAN) and/or wide area network (WAN), such as an intranet, an extranet, or the Internet. In various embodiments, the network 820 uses standard communication technologies and/or protocols. Examples of technologies used by the network 820 include Ethernet, 802.11, 3G, 4G, 802.16, or any other suitable communication technology. The network 820 may use wireless, wired, or a combination of wireless and wired communication technologies. Examples of networking protocols used for communicating via the network 820 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 820 may be represented using any suitable format, such as hypertext markup language (HTML) or extensible markup language (XML). In some embodiments, all or some of the communication links of the network 820 may be encrypted using any suitable technique or techniques.

The client device(s) 810 are computing devices capable of receiving user input as well as transmitting and/or receiving data via the network 820. In one embodiment, a client device 810 is a conventional computer system, such as a desktop or laptop computer. Alternatively, a client device 810 may be a device having computer functionality, such as a personal digital assistant (PDA), a mobile telephone, a smartphone or another suitable device. A client device 810 is configured to communicate via the network 820.

In some embodiments, the system environment 801 may include one or more servers, for example where the diagnostic system is includes a service that is managed by an entity that communicates via the network 820 with the reader 840 and/or any of the client devices 810. The server 830 can store data in database 805 and can access stored data in database 805. Database 805 may be an external database storing medical information, user or patient history data, etc. The server 830 may also store data in the cloud. In some embodiments, the server 830 may occasionally push updates to the reader 840, or may receive result data from the reader 840 and perform certain analyses on that result data and provide the analyzed data back to the reader 840 or to a client device 810.

In some embodiments, the reader 840 functionality can be included in a client device 810, such as a mobile phone, and can be operated via a mobile application installed on the phone. In these embodiments, a device may be attached to the phone that allows the phone to read the test strip, or the phone's own internal hardware (e.g., imaging hardware) can be used to read the test strip. The mobile application stored on the phone can process the results read from the test strip and share the results with other devices 810 on the network 820.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1: Application of a Coating Material on an Optical Tag Surface

Methods for applying a coating material to a silica surface of an optical tag may include the following procedure. A 1 milliliter (mL) aliquot of 0.5% 3-aminopropyltriethoxysilane (Aldrich Chemicals, Inc.) in ethanol is added to approximately 105 optical tags and shaken for 1 hour. The optical tags are washed with ethanol three times and once with acetonitrile in a centrifuge filter (Nanosep, 0.2 μm, Pall). Acetonitrile (980 μL), N,N-diisopropylethylamine (20 μL, Aldrich Chemicals, Inc.), and cyanuric chloride (10 mg, Aldrich Chemicals, Inc.) are added to the optical tags and the mixture is allowed to react for 2 hours with constant shaking.

The optical tags are then washed with acetonitrile 4 times, suspended in ethanol, and transferred to a microcentrifuge tube. The optical tags are settled by centrifugation at 4000 RPM and the supernatant decanted to a volume of approximately 25 μL.

Methods for applying a coating material to a silica surface of an optical tag may include the following procedure. The optical tags are incubated with a solution of 42 mM APTES in toluene for 1 hour. After the solution is removed, e.g., via filtration of the optical tags, the optical tags are washed with toluene, ethanol, and acetone and dried under a nitrogen stream (e.g. in a chamber). The APTES-modified optical tags are then immersed in a freshly prepared solution of 100 mg of succinic acid in 4.7 mL of DMSO and 300 μL of 0.1 M NaHC0 3, pH 9.4 for 30 minutes. After removal of the solution (e.g., by filtration of the optical tags), the optical tags were washed with DMSO two times and then washed with purified water.

### Example 2: Immobilization of a Reactive Moiety to an Optical Tag Surface

Reactive moieties of the disclosure may comprise an aptamer. Reactive moieties of the disclosure may be immobilized to a surface of the optical tag as follows. A 52 mM EDC solution is injected to a chamber containing the functionalized optical tags and allowed to react for 1 hour. Subsequently, 50 μL of 75 μM aptamer solution is applied to the optical tags for 1 hour, followed by thorough washing with 10 mL of 50 mM Tris buffer (e.g., through a filter with pores smaller than the optical tags) and a final folding of the aptamer by incubation in PBS for 30 minutes.

Optical tags may be oxidized prior to immobilization of a reactive moiety on a surface of the optical tag (either with or without a coating material to immobilize the reactive moiety). Oxidation, application of a coating material and/or immobilization of a reactive moiety may be performed on a film, which may be sonicated to form optical tags of the disclosure.

### Example 3: Detection of a Spectral Shift from a Unique Optical Tag to Determine the Presence of an Oligonucleotide

Silicon wafers were etched as described herein and in US Publication No. 2011/0147456, “Labeling and Authenticating Using a Microtag” to create a modulated porous structure in a film with reflectance peaks in the visible range. Using this process, two silicon films having a unique coded spectral response were generated. Both films were measured spectrally as described herein to generate a reference spectral signature, and the wavelengths of the peaks were recorded.

Each silicon film was functionalized with 5′ amino-modified probes using the method described in Steinberg et al, Biopolymers, Vol. 73, 597-605 (2004). Both films thus comprised a probe that binds specifically to the target analyte.

One film was incubated with a 10 nM solution of the target (complementary) analyte oligonucleotide at room temperature for 2.5 hours to allow hybridization between complementary oligonucleotides, as described in Meade et al., Anal Chem. 2009 April 1; 81(7): 2618-2625. The other film was incubated under the same conditions, but with a 10 nM solution of an alternate, non-complementary oligonucleotide. After incubation, both films were measured spectrally again, and the wavelengths of the peaks of the sample spectral signatures were recorded for each.

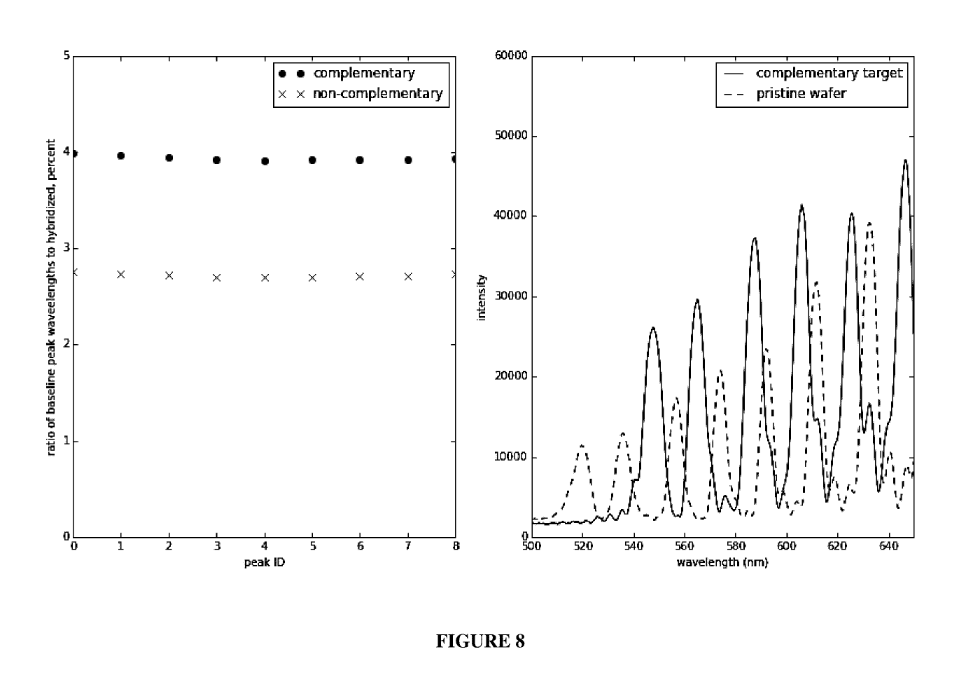

FIG. 8 (left) shows the peak shift for the spectral signature from each functionalized film measured after incubating the film with the target (complementary) analyte oligonucleotide (circle) or with a non-complementary oligonucleotide (x). The clear difference shows the ability of our functionalized tags to distinguish optical tags comprising analyte-bound probes from optical tags comprising unbound probes. FIG. 8 (right) also shows a reference spectrum from pristine wafer (dashed line) compared to a reference spectrum from a functionalized wafer bound to a target (complementary) oligonucleotide (solid line). This shows the clear shift in the spectral signature that can be used to determine both identity of an optical tag and a binding state.

### Other Embodiments

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of detecting an analyte suspected of being present in a sample, comprising:
a. providing a substrate comprising an optical tag immobilized on the surface of the substrate, wherein said optical tag is bound to a probe, wherein said optical tag comprises a plurality of pores, and wherein each of said plurality of pores comprises a modulated pore structure;
b. contacting said substrate with a sample suspected of comprising an analyte, wherein the probe is capable of binding specifically to the analyte;
c. exposing said optical tag to electromagnetic radiation to generate a sample spectral signature comprising at least one spectral feature that is a function of the modulated pore structure of the optical tag across a range of wavelengths;
d. detecting said sample spectral signature; and
e. comparing said sample spectral signature with a reference spectral signature to detect said analyte in said sample.

2. The method of claim 1, further comprising:
a. exposing said immobilized optical tag to electromagnetic radiation prior to contacting said substrate with said sample to generate a reference spectral signature comprising at least one spectral feature that is a function of the modulated pore structure of the optical tag across a range of wavelengths;
b. detecting said reference spectral signature; and
c. storing said reference spectral signature in a memory.

3. The method of claim 1, wherein the modulated pore structure across the plurality of pores in the optical tag is substantially similar.

4. The method of claim 2, wherein the reference spectral signature is determined from a functionalized or non-functionalized optical tag before contact with said sample.

5. The method of claim 2, wherein the reference spectral signature is directly measured or statistically determined.

6. The method of claim 5, wherein the statistically determined reference spectral signature is a function of a measured spectral signature from one or more optical tags with the same modulated pore structure, or is a function of the programmed properties of the modulated pore structure.

7. The method of claim 1, wherein said at least one spectral feature of said reference spectral signature is a rugate peak or a Bragg peak.

8. (canceled)

9. The method of claim 1, wherein said reference or sample spectral signature further comprises a Fabry-Perot spectral response.

10. The method of claim 1, wherein said spectral feature is a peak or a trough.

11. The method of claim 1, wherein said sample or reference spectral signature is linked to said optical tag by a spectral feature selected from the group consisting of: a unique peak number, a unique peak placement, a unique peak phase, a unique peak amplitude, a unique trough number, a unique trough placement, a unique trough phase, and a unique trough amplitude.

12. The method of claim 1, wherein comparing the sample spectral signature with the reference spectral signature comprises identifying the presence or absence of a spectral signature shift between the sample spectral signature and the reference spectral signature.

13. The method of claim 12, wherein detection of the spectral signature shift indicates the presence of the analyte in said sample.

14. The method of claim 12, wherein said spectral signature shift comprises a shift in a peak placement, peak phase, peak number, peak amplitude, trough placement, trough phase, trough number, or trough amplitude.

15. (canceled)

16. The method of claim 1, wherein said optical tag comprises silica or silicon.

17. The method of claim 16, wherein said optical tag is partially oxidized or fully oxidized.

18. The method of claim 1, wherein said optical tag comprises a non-silica dielectric.

19. The method of claim 1, wherein the optical tag comprises a stack of dielectric layers.

20. The method of claim 1, wherein said optical tag has a porosity of from 60 to 95%.

21. The method of claim 1, wherein said plurality of pores is sufficiently large to facilitate entry of said target analyte into said pores while excluding larger non-target molecules.

22. The method of claim 1, wherein said optical tag comprises a silica linker.

23. The method of claim 22, wherein said linker is an organofunctional alkoxysilane molecule.

24.-30. (canceled)

31. The method of claim 1, wherein the substrate comprises a plurality of unique optical tags immobilized on the surface of the substrate.

32. The method claim 31, wherein each of said unique optical tags is configured to generate a unique spectral signature comprising at least one peak or at least one trough that is a function of the modulated pore structure of the optical tag.

33. The method of claim 32, wherein said modulated pore structure is unique for each of said unique optical tags.

34. The method of claim 31, wherein the identity of said probe is correlated with said unique optical tag.

35. The method of claim 1, wherein detection of said reference or sample spectral signature comprises detecting an intensity at 20 or more, 30 or more, 40 or more, 50 or more, 60 or more, 70 or more, 80 or more, 90 or more, or 100 or more different narrow wavelength bands from a single one of said optical tags on the substrate.

36. The method of claim 1, wherein detection of said reference or sample spectral signature is performed using a device comprising an interferometer.

37. The method of claim 36, wherein said interferometer is a tunable Fabry-Perot interferometer or a Michelson type interferometer.

38. The method of claim 1, wherein detection of said reference or sample spectral signature comprises obtaining a wide spectral range from a field of view of about 100 mm².

39. The method of claim 1, wherein detection of said reference or sample spectral signature comprises placing said substrate in a portable device capable of obtaining a wide spectral range of said spectral signature.

40. The method of claim 39, wherein said portable device displays an identity of the analyte upon detection.

41. The method of claim 1, wherein the analyte does not comprise a detection label.

42. The method of claim 1, wherein the probe does not comprise a detection label.

43. The method of claim 1, wherein the method is label-free.

44. The method of claim 1, wherein said optical tag has a diameter, length, width, depth or height that is less than or equal to a millimeter.

45. The method of claim 1, wherein detecting said analyte in said sample comprises determining the presence or absence of said analyte in said sample.

46. The method claim 1, wherein detecting said analyte in said sample comprises determining a property of said analyte in said sample.

47. The method of claim 46, wherein said property is a concentration of said analyte, a binding affinity of said analyte to said probe, or a specific activity of said analyte.

48. The method of claim 47, wherein said substrate comprises a plurality of said optical tags, and wherein said concentration is determined from a proportion of said plurality of optical tags generating a shifted spectral signature, or is determined from a change in the average spectral signature for the plurality of optical tags.

49. The method of claim 1, further comprising performing a nucleic acid amplification reaction of said analyte before contacting said substrate with said sample, wherein said analyte is a polynucleotide.

50.-55. (canceled)

56. A method of detecting one or more analytes suspected of being present in a sample, comprising:
a. providing a plurality of optical tags each bound to at least one probe, wherein said plurality of optical tags each comprise a plurality of pores comprising a modulated pore structure;
b. contacting the plurality of optical tags with a sample suspected of comprising one or more analytes, wherein each probe is capable of binding specifically to one or more analytes or families of analytes;
c. immobilizing the plurality of optical tags on a surface of a substrate;
d. exposing each of the plurality of optical tags to electromagnetic radiation to generate a sample spectral signature for each of the plurality of optical tags, wherein the sample spectral signature comprises at least one spectral feature that is a function of the modulated pore structure of the optical tag across a range of wavelengths;
e. detecting said sample spectral signature for each of the plurality of optical tags; and
f. comparing said sample spectral signature with a reference spectral signature for each of said plurality of optical tags to detect said analyte in said sample.

57.-111. (canceled)

112. A composition comprising a substrate comprising a plurality of unique optical tags immobilized on the surface of the substrate, wherein each unique optical tag comprises a plurality of pores comprising a unique modulated pore structure, and wherein each unique optical tag is bound to a probe associated with said unique modulated pore structure.

113.-122. (canceled)

123. A diagnostic system for detecting the presence or absence of an analyte in a sample, comprising:
a. an optical tag bound to a probe, wherein said optical tag comprises a modulated pore structure, wherein said optical tag has been contacted with a sample suspected of containing an analyte; and
c. a reader device comprising:
one or more broadband sources configured to illuminate said optical tag;
a detector configured to detect reflected or transmitted light comprising a spectral response from said optical tag, wherein said spectral response comprises at least one spectral feature that is a function of the modulated pore structure of the optical tag across a range of wavelengths; and
a display configured to display results of the detection performed by the detector, the results indicating the presence or absence of the analyte in the sample.
